(19) United States
(12) Patent Application Publication
(10) Pub. No.: US 2025/0057844 A1
(43) Pub. Date: Feb. 20, 2025

(54) METHODS OF TREATING NEOPLASTIC DISEASES

(71) Applicant: SILLAJEN, INC., Seoul (KR)

(72) Inventors: Heidi LANE, Allschwil (CH); Felix BACHMANN, Allschwil (CH); Karine LITHERLAND, Allschwil (CH); Zaman GUIDO, Oss (NL)

(21) Appl. No.: 18/717,567

(22) PCT Filed: Dec. 14, 2022
(86) PCT No.: PCT/KR2022/020392

§ 371 Date: Jun. 7, 2024

(30) Foreign Application Priority Data
Dec. 15, 2021 (EP) 21214941.3
Aug. 22, 2022 (EP) 22191429.4

(51) Int. Cl. A61K 31/519; A61P 35/00

(52) U.S. Cl. CPC A61K 31/519; A61P 35/00

(57) ABSTRACT

The invention provides a compound of formula (I) or a pharmaceutically acceptable salt thereof as defined in the claims for use in the treatment of a neoplastic disease in a subject, wherein said treatment comprises administering the compound of formula (I) or a pharmaceutically acceptable salt thereof to the subject according to an intermittent dosing schedule.

### TECHNICAL FIELD

The present invention relates to methods of using the compound of formula (I) and pharmaceutically acceptable salts thereof as described herein in the treatment of neoplastic diseases, in particular cancer.

### BACKGROUND ART

WO 2015/155042 describes a recently discovered class of inhibitors of the threonine tyrosine kinase (TTK) for use in the treatment of cancer.

There is an ongoing need for new and effective treatment options for cancer patients. As demonstrated in the Examples below it has now surprisingly been found that in-termittent administration of a compound of formula (I) as described herein in cancer models provides superior efficacy in comparison to continuous dosing, including cures. In addition, it has also been found that the change from daily oral dosing to intermittent intravenous weekly dosing leads to a consistent drug exposure over time with an improved tolerability and gastrointestinal toxicity profiles in preclinical species.

### DISCLOSURE OF INVENTION

### Solution to Problem

### Summary of the Invention

In a first aspect the present invention provides a compound of formula (I)

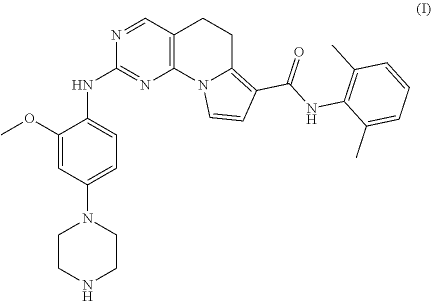 [SMILES: COc1cc(N2CCNCC2)ccc1Nc1ncc2c(n1)-n1ccc(C(=O)Nc3c(C)cccc3C)c1CC2]

or a pharmaceutically acceptable salt thereof;

for use in the treatment of a neoplastic disease in a subject, wherein the treatment comprises administering the compound of formula (I) or a pharmaceutically acceptable salt thereof to the subject according to an intermittent dosing schedule.

The compound of formula (I) is disclosed in WO 2015/155042 as Example 17.

It has also surprisingly been found that in addition to TTK inhibitory activity the compound of formula (I) also exhibits polo-like kinase 1 (PLK1) inhibition (see the Examples below). Both kinases collaborate in activating the mitotic spindle assembly checkpoint (SAC) at the kinetochore to regulate chromosome alignment and segregation before the cell can exit mitosis. Von Schubert et al., Cell Reports 2015, 12;66-78 discloses that PLK1 and TTK (also known as MPS1) cooperatively regulate the spindle assembly checkpoint (SAC) in human cells. This potential for an enhanced effect from inhibition of both TTK and PLKI is also alluded to in Dou et al., Plos ONE 2011, 6:4;e18793, which shows that some substrates of both kinases share a similar consensus motifs.

In contrast to TTK-specific inhibitors, the compound of formula (I) has a prolonged effect on TTK combined with a transient effect on PLKI (see Examples below) leading to a more rapid disruption of the SAC that potentiates aberrant mitotic progression. Accordingly, the dual TTK/PLKI inhibitory activity gives the compound of formula (I) a unique profile and differentiates it from other molecules which show TTK inhibitory activity without any appreciable levels of PLKI inhibitory activity.

In a further aspect the invention provides a method for treating a neoplastic disease in a subject in need thereof, wherein said treatment comprises administering a therapeutically effective amount of a compound of formula (I) or a pharmaceutically acceptable salt thereof to the subject according to an intermittent dosing schedule.

In a further aspect the invention provides use of a compound of formula (I) or a pharmaceutically acceptable salt thereof in the preparation of a medicament for the treatment of a neoplastic disease in a subject, wherein said treatment comprises administering a compound of formula (I) or a pharmaceutically acceptable salt thereof to the subject according to an intermittent dosing schedule.

Neoplastic diseases for treatment by the compound of formula (I) or a pharmaceutically acceptable salt thereof are described below, and are in particular contemplated for treatment of cancer, and in particular for human subjects.

Additional aspects and embodiments of the invention are described in more detail below.

### BRIEF DESCRIPTION OF FIGURES

FIG. 1: FIG. 1 shows the anti-tumor activity of weekly and twice-weekly in-termittent intravenous (i.v.) dosing of the compound of formula (I) in the triple negative breast cancer (TNBC) MDA-MB-231 model. The compound of formula (I) was administered i.v., QW or 2QW at the MTD or fractions thereof as indicated in the legend. (A) shows mean tumor volume, (B) shows mean percentage body weight changes until Day-65. Dashed vertical lines indicate a drug holiday for QW and 2QW MTD dosing groups. N=8 animals were used for treatment and vehicle control arms. Doxorubicin was administered using a standard dosing regimen of 3 mg/kg for the first 5 days. One non-drug related animal death occurred in the vehicle group and in each of the following dosing groups (3 mg/kg 2QW, 9.4 mg/kg 2QW, 12.5 mg/kg 2QW). Three non-drug-related animal deaths occurred in the 25 mg/kg QW dosing group.

FIG. 2: FIG. 2 shows the evaluation of cures with weekly MTD i.v. dosing in the TNBC MDA-MB-231 model. H&E sections of skin and surrounding tissue, obtained from the site of tumor implantation, were prepared from apparently tumor free animals (n=3) treated once-weekly with MTD i.v. dosing of the compound of formula (I) (animals 2, 3 and 8). In the samples from mouse 2 or mouse 3, no neoplastic epithelial cells (residual tumor cells) were observed, whereas mouse 8 still displayed some neoplastic tissue.

FIG. 3: FIG. 3 shows anti-tumor activity of daily oral MTD versus i.v. once-weekly intermittent dosing of the compound of formula (I) in the TNBC MDA-MB-231 model. The compound of formula (I) was administered orally daily at the MTD (7.5 mg/kg) or i.v. QW at the MTD or fractions thereof, as indicated in the legend. (A) shows mean tumor volume, (B) shows mean body weight changes in %. N=8 animals were used for treatment and vehicle control arms. No animal deaths were recorded.

FIG. 4: FIG. 4 shows anti-tumor activity of daily oral MTD vs i.v. twice-weekly intermittent dosing of the compound of formula (I) in the TNBC MDA-MB-231 model. The compound of formula (I) was administered orally daily at the MTD (7.5 mg/kg) or i.v. 2QW at the MTD or fractions thereof, as indicated in the legend. (A) shows mean tumor volume, (B) shows mean body weight changes in %. N=8 animals were used for treatment and vehicle control arms. No animal deaths were recorded.

FIG. 5: FIG. 5 shows the mean (+SD) plasma concentration-time profiles of the compound of formula (I) administered orally to female mice (n=3 animals per timepoint). (A) shows the plasma concentration on day 1 after single oral administration using formulation 2. (B) shows the plasma concentration on day 1 after single oral administration using formulation 1.

FIG. 6: FIG. 6 shows the mean (+SD) plasma concentration-time profiles of the compound of formula (I) administered orally to female mice (n=3 animals per timepoint). (A) shows the plasma concentration on day 5 after daily oral administration using formulation 1. (B) shows the plasma concentration on day 5 after daily oral administration using formulation 2.

FIG. 7: FIG. 7 shows the mean (+SD) plasma concentration-time profiles of the compound of formula (I) administered intravenously to female mice (n=3 animals per timepoint). (A) shows the plasma concentration on day 1 after single intravenous administration. (B) shows the plasma concentration on day 6 after oral administration for 5 days followed by a single intravenous administration on day 6.

### Detailed Description of the Invention

### Definitions

Certain terms used herein are described below. Unless defined otherwise, all technical and scientific terms used herein have the same meaning as is commonly un-derstood by one of skill in the art to which this invention belongs.

The term “pharmaceutically acceptable” as used herein refers to items such as compounds, materials, compositions and/or dosage forms, which are, within the scope of sound medical judgment, suitable for contact with the tissues of a human, without excessive toxicity or other complications commensurate with a reasonable benefit/risk ratio.

The term “patient” refers to a human presenting themselves for therapeutic treatment.

The term “subject” refers to a mammal and preferably refers to a patient.

The term “treatment,” as used herein in the context of treating a disease in a subject pertains generally to treatment and therapy in which some desired therapeutic effect is achieved, for example one or more of the following: the inhibition of the progress of the disease, a reduction in the rate of progress, a halt in the rate of progress, a prevention of the progression of the disease, alleviation of symptoms of the disease, amelioration of disease, and cure of the disease. For example, treatment can be the di-minishment of one or several symptoms of a disorder or complete eradication of a disorder. Within the meaning of the present disclosure, the term “treat” also denotes to arrest, delay the onset (i.e. the period prior to clinical manifestation of a disease) and/or reduce the risk of developing or worsening of a disease.

The term “prevent”, “preventing” or “prevention” as used herein comprises the prevention of at least one symptom associated with or caused by disease being prevented.

The term “pharmaceutically effective amount,” “therapeutically effective amount,” or “clinically effective amount” is an amount sufficient to provide an observable or clinically significant improvement over the baseline clinically observable signs and symptoms of the disease treated with the compound of formula (I) or a pharmaceutically acceptable salt thereof, e.g. commensurate with a reasonable benefit/risk ratio, when administered in accordance with a desired treatment regimen.

The term “about” means a variation of no more than 10% of the relevant figure. In some embodiments the term “about” means a variation of no more than 5% of the relevant figure.

For the avoidance of doubt, where a range is provided (e.g. 5 mg to 480 mg) the range includes the stated upper limit (480 mg) and lower limit (5 mg) of the range.

### Compounds of Formula (I) [59)

In some embodiments the compound of formula (I) is used as the free base. In other embodiments the compound of formula (I) is used as a pharmaceutically acceptable salt.

Pharmaceutically acceptable salts of the compound of formula (I) may be acid addition salts. Salts are formed, e.g. with organic or inorganic acids, from compounds of formula (I). Pharmaceutically acceptable salts are within the common general knowledge of the person skilled in the art. Pharmaceutically acceptable salts may include more than one molecule or ion of the corresponding acid.

The compounds of formula (I) and pharmaceutically acceptable salts thereof may be solvated, especially hydrated. Solvation and/or hydration may take place during the preparation process.

Compounds of formula (I) and pharmaceutically acceptable salts thereof may be syn-thesized as described in WO 2015/155042, in particular on pages 17 to 19 which are hereby incorporated by reference, and as described in Example 17 on page 49 of WO 2015/155042, which is also hereby incorporated by reference, including the reference in Example 17 to Example 9, Intermediate H and Example 1.

Diseases

The compound of formula (I) or a pharmaceutically acceptable salt thereof may be used to treat neoplastic diseases by administration of the compound of formula (I) or a pharmaceutically acceptable salt thereof, e.g. to inhibit the protein kinase. In addition in view of the observation that compounds of formula (I) additionally have PLKI inhibitory activity the neoplastic disease may be one which is treatable by inhibition of PLK1 in addition to a treatment with a TTK inhibitor (e.g. the compound of formula (I).

In addition, the compound of formula (I) or a pharmaceutically acceptable salt thereof may be used to treat a cancer at any clinical stage or pathological grade (e.g. tumor stage I, tumor stage II, tumor stage III, tumor stage IV) or treatment settings (e.g. preventative, adjuvant, neoadjuvant, therapeutic including palliative treatment). The compound of formula (I) or a pharmaceutically acceptable salt thereof may be for use in slowing, delaying or stopping cancer progression or cancer growth or increasing the overall survival time or the cancer-progression-free survival time or the time to progression of a cancer or improving or maintaining the subject's (e.g. patient's) quality of life or functional status. The compound of formula (I) or a pharmaceutically acceptable salt thereof may also be used in post-therapy recovery from cancer. The compound of formula (I) or a pharmaceutically acceptable salt thereof may be used in the treatment of metastatic cancer.

For example, the compound of formula (I) or a pharmaceutically acceptable salt thereof may be used for (i) reducing the number of cancer cells; (ii) reducing tumor volume; (iii) increasing tumor regression rate; (iv) reducing or slowing cancer cell in-filtration into peripheral organs; (v) reducing or slowing tumor metastasis; (vi) reducing or inhibiting tumor growth; (vii) preventing or delaying occurrence and/or re-currence of the cancer and/or extends disease-or tumor-free survival time; (viii) increasing overall survival time; (ix) reducing the frequency of treatment; and/or (x) relieving one or more of symptoms associated with the cancer.

As mentioned above, the compound of formula (I) or a pharmaceutically acceptable salt thereof may be used for the treatment of neoplastic diseases. Examples of neoplastic diseases include, but are not limited to, epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, naevi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, miscellaneous myeloproliferative disorders, lymphoproliferative disorders and myelodysplastic syndromes.

In some embodiments the neoplastic disease is cancer. Examples of cancers in terms of the organs and parts of the body affected include, but are not limited to, the brain, breast (including triple negative breast cancer and luminal B breast cancer), cervix, ovaries, colon, rectum (including colon and rectum i.e. colorectal cancer), lung (including small cell lung cancer, non-small cell lung cancer, large cell lung cancer and mesothelioma), endocrine system, bone, adrenal gland, thymus, liver, stomach, intestine (including gastric cancer), pancreas, bone marrow, hematological malignancies (such as lymphoma, leukemia, myeloma or lymphoid malignancies), bile duct, bladder, urinary tract, kidneys, skin, thyroid, head, neck, prostate and testis.

In some embodiments the neoplastic disease is a cancer selected from breast cancer (including triple negative breast cancer and luminal B breast cancer), gastric cancer, colorectal cancer, liver cancer (including hepatocellular cancer), endometrial cancer, ovarian cancer, esophageal cancer, lung cancer (including small cell lung cancer, non-small cell lung cancer), Kaposi's sarcoma, cervical cancer, pancreatic cancer, melanoma, prostate cancer, testicular cancer, cervical cancer, bladder cancer, head and neck cancer, brain tumor (e.g. glioma, medulloblastoma), neuroblastoma, retinoblastoma, Wilms' tumor, leukemia, e.g. acute myeloid leukemia (AML) (including Complex Karyotype AML) and malignant mesothelioma.

In some embodiments the neoplastic disease is breast cancer.

In some embodiments the neoplastic disease is triple negative breast cancer.

In some embodiments the neoplastic disease is luminal B breast cancer.

In some embodiments the neoplastic disease is gastric cancer.

In some embodiments the neoplastic disease is colorectal cancer.

In some embodiments the neoplastic disease is hepatocellular cancer.

In some embodiments the neoplastic disease is endometrial cancer.

In some embodiments the neoplastic disease is acute myeloid leukemia (AML) (including Complex Karyotype AML).

In some embodiments the neoplastic disease is lung cancer (e.g. small cell lung cancer, non-small cell lung cancer).

In some embodiments the neoplastic disease is cervical cancer (e.g. metastatic or recurrent cervical cancer).

In some embodiments the neoplastic disease is head and neck cancer (e.g. recurrent or metastatic squamous cell carcinoma of the head and neck).

In some embodiments the neoplastic disease is Wilms' tumor.

In some embodiments the neoplastic disease is a brain tumor (e.g. gliomas, such as progressive or recurrent gliomas, medulloblastoma, such as recurrent medulloblastoma).

In some embodiments the neoplastic disease is neuroblastoma.

In some embodiments the neoplastic disease is testicular cancer (e.g. metastatic non-seminomatous germ cell tumor).

In some embodiments the neoplastic disease is bladder cancer (e.g. advanced bladder cancer, including those with abnormal renal function).

In some embodiments the neoplastic disease is retinoblastoma (e.g. recurrent or progressive retinoblastoma.

The cancer may be a primary tumor and/or metastases. The cancer may be derived from a solid or liquid (e.g. hematological or intraperitoneal) tumor. In some embodiments the neoplastic disease (e.g. cancer) to be treated is a tumor, e.g. a solid tumor.

### Administration

The compound of formula (I) or a pharmaceutically acceptable salt thereof is administered according to an intermittent dosing schedule. An intermittent dosing schedule is one which comprises intervals of more than one day between scheduled doses. An intermittent dosing schedule is different to a continuous dosing schedule in which the subject is dosed every day. Preferably the compound of formula (I) or a pharmaceutically acceptable salt thereof is administered to the subject (preferably a human) intravenously.

In some embodiments the intermittent dosing schedule comprises an interval of at least 2 days, e.g. at least 3 days, e.g. at least 4 days, e.g. at least 5 days e.g. at least 6 days, e.g. at least 7 days during which no compound of formula (I) or a pharmaceutically acceptable salt thereof is administered to the subject.

A subject may receive a scheduled dose in one administration or in more than one administration, e.g. on the same day. For example, half the scheduled dose may be administered in the morning and the second half in the afternoon etc. After the full scheduled dose has been administered there is then an interval of more than one day before administration of the next scheduled dose. Accordingly, in some embodiments there is an interval of at least 2 days, e.g. at least 3 days, e.g. at least 4 days, e.g. at least 5 days e.g. at least 6 days, e.g. at least 7 days between consecutive scheduled doses. In some embodiments there is an interval of at least 2 days, e.g. at least 3 days, e.g. at least 4 days, e.g. at least 5 days e.g. at least 6 days, e.g. at least 7 days between each consecutive scheduled dose.

The intervals between scheduled doses may be regular (e.g. intervals of the same number of days) or irregular (e.g. of intervals of different numbers of days), e.g. depending on the response of the subject to the drug.

It is also contemplated that a subject may receive a scheduled dose in one administration, i.e. a single administration without any pauses. Accordingly, in some embodiments there is an interval of at least 2 days, e.g. at least 3 days, e.g. at least 4 days, e.g. at least 5 days e.g. at least 6 days, e.g. at least 7 days between consecutive administrations. In some embodiments there is an interval of at least 2 days, e.g. at least 3 days, e.g. at least 4 days, e.g. at least 5 days e.g. at least 6 days, e.g. at least 7 days between each consecutive administration.

The intermittent dosing schedule will usually be a cyclic treatment schedule. A cyclic treatment schedule is defined by a repeated dosing schedule wherein the repeated element (a cycle) has a specific duration and wherein doses are administered on specific days within the cycle. A cycle may incorporate a period, usually at the end of the cycle, in which there is no administration (a “rest period”), e.g. to allow a period for recovery. A treatment cycle may be, e.g. 7 days, 14 days, 21 days, 28 days or longer. The treatment schedule may be continued for as long as required (an “open-end treatment”) e.g. as long as the subject (e.g. patient) is receiving benefit judged by a physician overseeing the treatment.

In some embodiments the compound of formula (I) or a pharmaceutically acceptable salt thereof is administered according to a three week treatment cycle, wherein the compound of formula (I) or a pharmaceutically acceptable salt thereof is administered in week one, e.g. on day 1, of the treatment cycle followed by two rest weeks.

In some embodiments the compound of formula (I) or a pharmaceutically acceptable salt thereof is administered according to a three week treatment cycle, wherein the compound of formula (I) or a pharmaceutically acceptable salt thereof is administered in week one and week two of the treatment cycle, e.g. on day 1 and day 8, followed by one rest week.

In some embodiments the compound of formula (I) or a pharmaceutically acceptable salt thereof is administered according to a four week treatment cycle, wherein the compound of formula (I) or a pharmaceutically acceptable salt thereof is administered in week one of the treatment cycle, e.g. on day 1, followed by three rest weeks.

In some embodiments the compound of formula (I) or a pharmaceutically acceptable salt thereof is administered according to a four week treatment cycle, wherein the compound of formula (I) or a pharmaceutically acceptable salt thereof is administered in week one and in week three of the treatment cycle, e.g. on day 1 and day 15, with weeks two and four being rest weeks.

In some embodiments the compound of formula (I) or a pharmaceutically acceptable salt thereof is administered according to a four week treatment cycle, wherein the compound of formula (I) or a pharmaceutically acceptable salt thereof is administered in each week during the first three weeks of the treatment cycle, e.g. on day 1, day 8 and day 15, followed by a rest week.

The following examples of dosages are for humans. The doses of compound of formula (I) are given as mg per person irrespective of body weight or body surface area (BSA). Dosages of the compound of formula (I) as given below, including in Tables A and B, refer to dosages of the free base. The dosages also apply to pharmaceutically acceptable salts of the compound of formula (I), except that when a pharmaceutically acceptable salt of the compound of formula (I) is used the stated mg dosage amount should be adjusted (i.e. increased) so that the molar amount of the pharmaceutically acceptable salt of the compound of formula (I) to be dosed is the same as the molar amount of the free base as given below. For example, a statement that the (human) weekly dosage amount of the compound of formula (I) is about 5 mg to about 480 mg in weeks when administered means that the compound of formula (I) or a pharmaceutically acceptable salt thereof is administered to a patient at a dose corresponding to the mole equivalent of about 5 mg to about 480 mg of the free base of the compound of formula (I) per week in weeks when administered.

In some embodiments the (human) weekly dosage amount of the compound of formula (I) is about 5 mg to about 480 mg in weeks when administered. In some embodiments the (human) weekly dosage amount of the compound of formula (I) is about 40 mg to about 200 mg in weeks when administered. In some embodiments the (human) weckly dosage amount of the compound of formula (I) is about 80 mg to about 160 mg in weeks when administered. In some embodiments the (human) weckly dosage amount of the compound of formula (I) is about 90 mg to about 130 mg in weeks when administered.

In some embodiments the (human) weekly dosage amount of the compound of formula (I) is about 140 mg to about 240 mg in weeks when administered. In some embodiments the (human) weekly dosage amount of the compound of formula (I) is about 160 mg to about 220 mg in weeks when administered. In some embodiments the (human) weekly dosage amount of the compound of formula (I) is about 180 mg to about 200 mg in weeks when administered.

Examples of (human) weekly dosage amounts of the compound of formula (I) in weeks when administered include about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, about 160 mg to about 170 mg, about 170 mg to about 180 mg, about 180 mg to about 190 mg, about 190 mg to about 200 mg, about 200 mg to about 210 mg, about 210 mg to about 220 mg, about 220 mg to about 230 mg, about 230 mg to about 240 mg, about 240 mg to about 250 mg, about 250 mg to about 260 mg, about 260 mg to about 270 mg, about 270 mg to about 280 mg, about 280 mg to about 290 mg, about 290 mg to about 300 mg, about 300 mg to about 310 mg, about 310 mg to about 320 mg, about 320 mg to about 330 mg, about 330 mg to about 340 mg, about 340 mg to about 350 mg, about 350 mg to about 360 mg, about 360 mg to about 370 mg, about 370 mg to about 380 mg, about 380 mg to about 390 mg, about 390 mg to about 400 mg, about 400 mg to about 410 mg, about 410 mg to about 420 mg, about 420 mg to about 430 mg, about 430 mg to about 440 mg, about 440 mg to about 450 mg, about 450 mg to about 460 mg, about 460 mg to about 470 mg, and about 470 mg to about 480 mg.

Examples of specific (human) weekly dosage amounts of the compound of formula (I) in weeks when administered include about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, and about 480 mg.

The weekly dose of the compound of formula (I) or a pharmaceutically acceptable salt thereof may be administered in a single administration, e.g. without any pause when administered intravenously. Alternatively the weekly dose may be administered in multiple administrations, e.g. in two or three administrations with pauses in between administrations when administered intravenously, e.g. of at least 30 minutes, e.g. at least an hour, e.g. at least two hours, e.g. at least 4 hours between administrations, e.g. 30 minutes to 12 hours, e.g. 30 minutes to 6 hours between administrations. Such multiple administrations may be on the same day or on separate days, e.g. on consecutive days or e.g. on the third day after the day of initial administration, providing that the dosing schedule is an intermittent dosing schedule as described above.

The compound of formula (I) or a pharmaceutically acceptable salt thereof may be administered to the subject according to the usual routes of administration known by the person skilled in the art, but is preferably administered to the subject intravenously. The duration of the infusion will usually be at least 30 minutes and may be up to 24 hours. In some embodiments the duration of the infusion is 30 minutes to 12 hours, e.g. 30 minutes to 6 hours, e.g. 30 minutes to 3 hours, e.g. one to two hours, e.g. about one hour.

In some embodiments the treatment cycle duration, the weeks of administration and the (human) weekly dosage of the compound of formula (I) is as indicated in any one of embodiments 1A to 35A in Table A, wherein the compound of formula (I) or a pharmaceutically acceptable salt thereof is preferably administered intravenously to the patient.

TABLE A

| | Cycle | Compound of formula (I) | |
|---|---|---|---|
| | duration | Weeks of | Weekly |
| Embodiment | (weeks) | administration | dosage (mg) |
| 1A | 3 | 1 | 5-480 |
| 2A | 3 | 1 | 40-200 |
| 3A | 3 | 1 | 80-160 |
| 4A | 3 | 1 | 90-130 |
| 5A | 3 | 1 | 140-240 |
| 6A | 3 | 1 | 160-220 |
| 7A | 3 | 1 | 180-200 |
| 8A | 3 | 1, 2 | 5-480 |
| 9A | 3 | 1, 2 | 40-200 |
| 10A | 3 | 1, 2 | 80-160 |
| 11A | 3 | 1, 2 | 90-130 |
| 12A | 3 | 1, 2 | 140-240 |
| 13A | 3 | 1, 2 | 160-220 |
| 14A | 3 | 1, 2 | 180-200 |
| 15A | 4 | 1 | 5-480 |
| 16A | 4 | 1 | 40-200 |
| 17A | 4 | 1 | 80-160 |
| 18A | 4 | 1 | 90-130 |
| 19A | 4 | 1 | 140-240 |
| 20A | 4 | 1 | 160-220 |
| 21A | 4 | 1 | 180-200 |
| 22A | 4 | 1, 2 | 5-480 |
| 23A | 4 | 1, 2 | 40-200 |
| 24A | 4 | 1, 2 | 80-160 |
| 25A | 4 | 1, 2 | 90-130 |
| 26A | 4 | 1, 2 | 140-240 |
| 27A | 4 | 1, 2 | 160-220 |
| 28A | 4 | 1, 2 | 180-200 |
| 29A | 4 | 1, 2, 3 | 5-480 |
| 30A | 4 | 1, 2, 3 | 40-200 |
| 31A | 4 | 1, 2, 3 | 80-160 |
| 32A | 4 | 1, 2, 3 | 90-130 |
| 33A | 4 | 1, 2, 3 | 140-240 |
| 34A | 4 | 1, 2, 3 | 160-220 |
| 35A | 4 | 1, 2, 3 | 180-200 |

As per the statement above, embodiment 1A refers to the situation wherein the compound of formula (I) or a pharmaceutically acceptable salt thereof is administered to a patient according to a three week treatment cycle, wherein the compound of formula (I) or a pharmaceutically acceptable salt thereof is administered in week one of the treatment cycle followed by two rest weeks and wherein the compound of formula (I) or a pharmaceutically acceptable salt thereof is administered to a patient at a dose corresponding to the mole equivalent of about 5 mg to about 480 mg of the free base of the compound of formula (I) per week in weeks when administered. The same applies analogously to embodiments 2A to 32A.

In some embodiments the treatment cycle duration, the days of administration within the cycle and the (human) weekly dosage of the compound of formula (I) is as indicated in any one of embodiments 1B to 35B in Table B, wherein the compound of formula (I) or a pharmaceutically acceptable salt thereof is preferably administered intravenously to the patient.

TABLE B

| | Cycle | Compound of formula (I) | |
|---|---|---|---|
| | duration | Days of | Weekly |
| Embodiment | (days) | administration | dosage (mg) |
| 1B | 21 | 1 | 5-480 |
| 2B | 21 | 1 | 40-200 |
| 3B | 21 | 1 | 80-160 |
| 4B | 21 | 1 | 90-130 |
| 5B | 21 | 1 | 140-240 |
| 6B | 21 | 1 | 160-220 |
| 7B | 21 | 1 | 180-200 |
| 8B | 21 | 1, 8 | 5-480 |
| 9B | 21 | 1, 8 | 40-200 |
| 10B | 21 | 1, 8 | 80-160 |
| 11B | 21 | 1, 8 | 90-130 |
| 12B | 21 | 1, 8 | 140-240 |
| 13B | 21 | 1, 8 | 160-220 |
| 14B | 21 | 1, 8 | 180-200 |
| 15B | 28 | 1 | 5-480 |
| 16B | 28 | 1 | 40-200 |
| 17B | 28 | 1 | 80-160 |
| 18B | 28 | 1 | 90-130 |
| 19B | 28 | 1 | 140-240 |
| 20B | 28 | 1 | 160-220 |
| 21B | 28 | 1 | 180-200 |
| 22B | 28 | 1, 8 | 5-480 |
| 23B | 28 | 1, 8 | 40-200 |
| 24B | 28 | 1, 8 | 80-160 |
| 25B | 28 | 1, 8 | 90-130 |
| 26B | 28 | 1, 8 | 140-240 |
| 27B | 28 | 1, 8 | 160-220 |
| 28B | 28 | 1, 8 | 180-200 |
| 29B | 28 | 1, 8, 15 | 5-480 |
| 30B | 28 | 1, 8, 15 | 40-200 |
| 31B | 28 | 1, 8, 15 | 80-160 |
| 32B | 28 | 1, 8, 15 | 90-130 |
| 33B | 28 | 1, 8, 15 | 140-240 |
| 34B | 28 | 1, 8, 15 | 160-220 |
| 35B | 28 | 1, 8, 15 | 180-200 |

As per the statement above, embodiment 1B refers to the situation wherein the compound of formula (I) or a pharmaceutically acceptable salt thereof is administered to a patient according to a 21 day treatment cycle, wherein the compound of formula (I) or a pharmaceutically acceptable salt thereof is administered on day 1 of the treatment cycle and wherein the compound of formula (I) or a pharmaceutically acceptable salt thereof is administered to a patient at a dose corresponding to the mole equivalent of about 5 mg to about 480 mg of the free base of the compound of formula (I) per week in weeks when administered. The same applies analogously to embodiments 2B to 32B.

In some embodiments the treatment cycle duration, the weeks of administration, the (human) weekly dosage and the neoplastic disease is as indicated in any one of embodiments 1C to 210C in Table C, wherein the compound of formula (I) or a pharmaceutically acceptable salt thereof is preferably administered intravenously to the patient.

TABLE C

| | Cycle | Compound of formula (I) | | |
|---|---|---|---|---|
| | duration | Weeks of | Weekly | Neoplastic |
| Embodiment | (weeks) | administration | dosage (mg) | disease |
| 1C | 3 | 1 | 5-480 | BC |
| 2C | 3 | 1 | 40-200 | BC |
| 3C | 3 | 1 | 80-160 | BC |
| 4C | 3 | 1 | 90-130 | BC |
| 5C | 3 | 1 | 140-240 | BC |
| 6C | 3 | 1 | 160-220 | BC |
| 7C | 3 | 1 | 180-200 | BC |
| 8C | 3 | 1, 2 | 5-480 | BC |
| 9C | 3 | 1, 2 | 40-200 | BC |
| 10C | 3 | 1, 2 | 80-160 | BC |
| 11C | 3 | 1, 2 | 90-130 | BC |
| 12C | 3 | 1, 2 | 140-240 | BC |
| 13C | 3 | 1, 2 | 160-220 | BC |
| 14C | 3 | 1, 2 | 180-200 | BC |
| 15C | 4 | 1 | 5-480 | BC |
| 16C | 4 | 1 | 40-200 | BC |
| 17C | 4 | 1 | 80-160 | BC |
| 18C | 4 | 1 | 90-130 | BC |
| 19C | 4 | 1 | 140-240 | BC |
| 20C | 4 | 1 | 160-220 | BC |
| 21C | 4 | 1 | 180-200 | BC |
| 22C | 4 | 1, 2 | 5-480 | BC |
| 23C | 4 | 1, 2 | 40-200 | BC |
| 24C | 4 | 1, 2 | 80-160 | BC |
| 25C | 4 | 1, 2 | 90-130 | BC |
| 26C | 4 | 1, 2 | 140-240 | BC |
| 27C | 4 | 1, 2 | 160-220 | BC |
| 28C | 4 | 1, 2 | 180-200 | BC |
| 29C | 4 | 1, 2, 3 | 5-480 | BC |
| 30C | 4 | 1, 2, 3 | 40-200 | BC |
| 31C | 4 | 1, 2, 3 | 80-160 | BC |
| 32C | 4 | 1, 2, 3 | 90-130 | BC |
| 33C | 4 | 1, 2, 3 | 140-240 | BC |
| 34C | 4 | 1, 2, 3 | 160-220 | BC |
| 35C | 4 | 1, 2, 3 | 180-200 | BC |
| 36C | 3 | 1 | 5-480 | GC |
| 37C | 3 | 1 | 40-200 | GC |
| 38C | 3 | 1 | 80-160 | GC |
| 39C | 3 | 1 | 90-130 | GC |
| 40C | 3 | 1 | 140-240 | GC |
| 41C | 3 | 1 | 160-220 | GC |
| 42C | 3 | 1 | 180-200 | GC |
| 43C | 3 | 1, 2 | 5-480 | GC |
| 44C | 3 | 1, 2 | 40-200 | GC |
| 45C | 3 | 1, 2 | 80-160 | GC |
| 46C | 3 | 1, 2 | 90-130 | GC |
| 47C | 3 | 1, 2 | 140-240 | GC |
| 48C | 3 | 1, 2 | 160-220 | GC |
| 49C | 3 | 1, 2 | 180-200 | GC |
| 50C | 4 | 1 | 5-480 | GC |
| 51C | 4 | 1 | 40-200 | GC |
| 52C | 4 | 1 | 80-160 | GC |
| 53C | 4 | 1 | 90-130 | GC |
| 54C | 4 | 1 | 140-240 | GC |
| 55C | 4 | 1 | 160-220 | GC |
| 56C | 4 | 1 | 180-200 | GC |
| 57C | 4 | 1, 2 | 5-480 | GC |
| 58C | 4 | 1, 2 | 40-200 | GC |
| 59C | 4 | 1, 2 | 80-160 | GC |
| 60C | 4 | 1, 2 | 90-130 | GC |
| 61C | 4 | 1, 2 | 140-240 | GC |
| 62C | 4 | 1, 2 | 160-220 | GC |
| 63C | 4 | 1, 2 | 180-200 | GC |
| 64C | 4 | 1, 2, 3 | 5-480 | GC |
| 65C | 4 | 1, 2, 3 | 40-200 | GC |
| 66C | 4 | 1, 2, 3 | 80-160 | GC |
| 67C | 4 | 1, 2, 3 | 90-130 | GC |
| 68C | 4 | 1, 2, 3 | 140-240 | GC |
| 69C | 4 | 1, 2, 3 | 160-220 | GC |
| 70C | 4 | 1, 2, 3 | 180-200 | GC |
| 71C | 3 | 1 | 5-480 | CC |
| 72C | 3 | 1 | 40-200 | CC |
| 73C | 3 | 1 | 80-160 | CC |
| 74C | 3 | 1 | 90-130 | CC |
| 75C | 3 | 1 | 140-240 | CC |
| 76C | 3 | 1 | 160-220 | CC |
| 77C | 3 | 1 | 180-200 | CC |
| 78C | 3 | 1, 2 | 5-480 | CC |
| 79C | 3 | 1, 2 | 40-200 | CC |
| 80C | 3 | 1, 2 | 80-160 | CC |
| 81C | 3 | 1, 2 | 90-130 | CC |
| 82C | 3 | 1, 2 | 140-240 | CC |
| 83C | 3 | 1, 2 | 160-220 | CC |
| 84C | 3 | 1, 2 | 180-200 | CC |
| 85C | 4 | 1 | 5-480 | CC |
| 86C | 4 | 1 | 40-200 | CC |
| 87C | 4 | 1 | 80-160 | CC |
| 88C | 4 | 1 | 90-130 | CC |
| 89C | 4 | 1 | 140-240 | CC |
| 90C | 4 | 1 | 160-220 | CC |
| 91C | 4 | 1 | 180-200 | CC |
| 92C | 4 | 1, 2 | 5-180 | CC |
| 93C | 4 | 1, 2 | 40-200 | CC |
| 94C | 4 | 1, 2 | 80-160 | CC |
| 95C | 4 | 1, 2 | 90-130 | CC |
| 96C | 4 | 1, 2 | 140-240 | CC |
| 97C | 4 | 1, 2 | 160-220 | CC |
| 98C | 4 | 1, 2 | 180-200 | CC |
| 99C | 4 | 1, 2, 3 | 5-480 | CC |
| 100C | 4 | 1, 2, 3 | 40-200 | CC |
| 101C | 4 | 1, 2, 3 | 80-160 | CC |
| 102C | 4 | 1, 2, 3 | 90-130 | CC |
| 103C | 4 | 1, 2, 3 | 140-240 | CC |
| 104C | 4 | 1, 2, 3 | 160-220 | CC |
| 105C | 4 | 1, 2, 3 | 180-200 | CC |
| 106C | 3 | 1 | 5-480 | HC |
| 107C | 3 | 1 | 40-200 | HC |
| 108C | 3 | 1 | 80-160 | HC |
| 109C | 3 | 1 | 90-130 | HC |
| 110C | 3 | 1 | 140-240 | HC |
| 111C | 3 | 1 | 160-220 | HC |
| 112C | 3 | 1 | 180-200 | HC |
| 113C | 3 | 1, 2 | 5-480 | HC |
| 114C | 3 | 1, 2 | 40-200 | HC |
| 115C | 3 | 1, 2 | 80-160 | HC |
| 116C | 3 | 1, 2 | 90-130 | HC |
| 117C | 3 | 1, 2 | 140-240 | HC |
| 118C | 3 | 1, 2 | 160-220 | HC |
| 119C | 3 | 1, 2 | 180-200 | HC |
| 120C | 4 | 1 | 5-480 | HC |
| 121C | 4 | 1 | 40-200 | HC |
| 122C | 4 | 1 | 80-160 | HC |
| 123C | 4 | 1 | 90-130 | HC |
| 124C | 4 | 1 | 140-240 | HC |
| 125C | 4 | 1 | 160-220 | HC |
| 126C | 4 | 1 | 180-200 | HC |
| 127C | 4 | 1, 2 | 5-480 | HC |
| 128C | 4 | 1, 2 | 40-200 | HC |
| 129C | 4 | 1, 2 | 80-160 | HC |
| 130C | 4 | 1, 2 | 90-130 | HC |
| 131C | 4 | 1, 2 | 140-240 | HC |
| 132C | 4 | 1, 2 | 160-220 | HC |
| 133C | 4 | 1, 2 | 180-200 | HC |
| 134C | 4 | 1, 2, 3 | 5-480 | HC |
| 135C | 4 | 1, 2, 3 | 40-200 | HC |
| 136C | 4 | 1, 2, 3 | 80-160 | HC |
| 137C | 4 | 1, 2, 3 | 90-130 | HC |
| 138C | 4 | 1, 2, 3 | 140-240 | HC |
| 139C | 4 | 1, 2, 3 | 160-220 | HC |
| 140C | 4 | 1, 2, 3 | 180-200 | HC |
| 141C | 3 | 1 | 5-480 | EC |
| 142C | 3 | 1 | 40-200 | EC |
| 143C | 3 | 1 | 80-160 | EC |
| 144C | 3 | 1 | 90-130 | EC |
| 145C | 3 | 1 | 140-240 | EC |
| 146C | 3 | 1 | 160-220 | EC |
| 147C | 3 | 1 | 180-200 | EC |
| 148C | 3 | 1, 2 | 5-480 | EC |
| 149C | 3 | 1, 2 | 40-200 | EC |
| 150C | 3 | 1, 2 | 80-160 | EC |
| 151C | 3 | 1, 2 | 90-130 | EC |
| 152C | 3 | 1, 2 | 140-240 | EC |
| 153C | 3 | 1, 2 | 160-220 | EC |
| 154C | 3 | 1, 2 | 180-200 | EC |
| 155C | 4 | 1 | 5-480 | EC |
| 156C | 4 | 1 | 40-200 | EC |
| 157C | 4 | 1 | 80-160 | EC |
| 158C | 4 | 1 | 90-130 | EC |
| 159C | 4 | 1 | 140-240 | EC |
| 160C | 4 | 1 | 160-220 | EC |
| 161C | 4 | 1 | 180-200 | EC |
| 162C | 4 | 1, 2 | 5-480 | EC |
| 163C | 4 | 1, 2 | 40-200 | EC |
| 164C | 4 | 1, 2 | 80-160 | EC |
| 165C | 4 | 1, 2 | 90-130 | EC |
| 166C | 4 | 1, 2 | 140-240 | EC |
| 167C | 4 | 1, 2 | 160-220 | EC |
| 168C | 4 | 1, 2 | 180-200 | EC |
| 169C | 4 | 1, 2, 3 | 5-480 | EC |
| 170C | 4 | 1, 2, 3 | 40-200 | EC |
| 171C | 4 | 1, 2, 3 | 80-160 | EC |
| 172C | 4 | 1, 2, 3 | 90-130 | EC |
| 173C | 4 | 1, 2, 3 | 140-240 | EC |
| 174C | 4 | 1, 2, 3 | 160-220 | EC |
| 175C | 4 | 1, 2, 3 | 180-200 | EC |
| 176C | 3 | 1 | 5-480 | AML |
| 177C | 3 | 1 | 40-200 | AML |
| 178C | 3 | 1 | 80-160 | AML |
| 179C | 3 | 1 | 90-130 | AML |
| 180C | 3 | 1 | 140-240 | AML |
| 181C | 3 | 1 | 160-220 | AML |
| 182C | 3 | 1 | 180-200 | AML |
| 183C | 3 | 1, 2 | 5-480 | AML |
| 184C | 3 | 1, 2 | 40-200 | AML |
| 185C | 3 | 1, 2 | 80-160 | AML |
| 186C | 3 | 1, 2 | 90-130 | AML |
| 187C | 3 | 1, 2 | 140-240 | AML |
| 188C | 3 | 1, 2 | 160-220 | AML |
| 189C | 3 | 1, 2 | 180-200 | AML |
| 190C | 4 | 1 | 5-480 | AML |
| 191C | 4 | 1 | 40-200 | AML |
| 192C | 4 | 1 | 80-160 | AML |
| 193C | 4 | 1 | 90-130 | AML |
| 194C | 4 | 1 | 140-240 | AML |
| 195C | 4 | 1 | 160-220 | AML |
| 196C | 4 | 1 | 180-200 | AML |
| 197C | 4 | 1, 2 | 5-480 | AML |
| 198C | 4 | 1, 2 | 40-200 | AML |
| 199C | 4 | 1, 2 | 80-160 | AML |
| 200C | 4 | 1, 2 | 90-130 | AML |
| 201C | 4 | 1, 2 | 140-240 | AML |
| 202C | 4 | 1, 2 | 160-220 | AML |
| 203C | 4 | 1, 2 | 180-200 | AML |
| 204C | 4 | 1, 2, 3 | 5-480 | AML |
| 205C | 4 | 1, 2, 3 | 40-200 | AML |
| 206C | 4 | 1, 2, 3 | 80-160 | AML |
| 207C | 4 | 1, 2, 3 | 90-130 | AML |
| 208C | 4 | 1, 2, 3 | 140-240 | AML |
| 209C | 4 | 1, 2, 3 | 160-220 | AML |
| 210C | 4 | 1, 2, 3 | 180-200 | AML |

BC = Breast cancer (e.g. triple negative breast cancer or luminal B breast cancer, in particular triple negative breast cancer),
GC = gastric cancer,
CC = colorectal cancer,
HC = hepatocellular cancer,
EC = endometrial cancer,
AML = acute myeloid leukemia (e.g. Complex Karyotype AML).

As per the statement above, embodiment 1C refers to the situation wherein the neoplastic disease to be treated is breast cancer and compound of formula (I) or a pharmaceutically acceptable salt thereof is administered to a patient according to a three week treatment cycle, wherein the compound of formula (I) or a pharmaceutically acceptable salt thereof is administered in week one of the treatment cycle followed by two rest weeks and wherein the compound of formula (I) or a pharmaceutically acceptable salt thereof is administered to a patient at a dose corresponding to the mole equivalent of about 5 mg to about 480 mg of the free base of the compound of formula (I) per week in weeks when administered. The same applies analogously to embodiments 2C to 210C.

In some embodiments the treatment cycle duration, the days of administration within the cycle, the (human) weekly dosage and the neoplastic disease is as indicated in any one of embodiments 1D to 210D in Table D, wherein the compound of formula (I) or a pharmaceutically acceptable salt thereof is preferably administered intravenously to the patient.

TABLE D

| | Cycle | Compound of formula (I) | | |
|---|---|---|---|---|
| | duration | Days of | Weekly | Neoplastic |
| Embodiment | (days) | administration | dosage (mg) | disease |
| 1D | 21 | 1 | 5-480 | BC |
| 2D | 21 | 1 | 40-200 | BC |
| 3D | 21 | 1 | 80-160 | BC |
| 4D | 21 | 1 | 90-130 | BC |
| 5D | 21 | 1 | 140-240 | BC |
| 6D | 21 | 1 | 160-220 | BC |
| 7D | 21 | 1 | 180-200 | BC |
| 8D | 21 | 1, 8 | 5-480 | BC |
| 9D | 21 | 1, 8 | 40-200 | BC |
| 10D | 21 | 1, 8 | 80-160 | BC |
| 11D | 21 | 1, 8 | 90-130 | BC |
| 12D | 21 | 1, 8 | 140-240 | BC |
| 13D | 21 | 1, 8 | 160-220 | BC |
| 14D | 21 | 1, 8 | 180-200 | BC |
| 15D | 28 | 1 | 5-480 | BC |
| 16D | 28 | 1 | 40-200 | BC |
| 17D | 28 | 1 | 80-160 | BC |
| 18D | 28 | 1 | 90-130 | BC |
| 19D | 28 | 1 | 140-240 | BC |
| 20D | 28 | 1 | 160-220 | BC |
| 21D | 28 | 1 | 180-200 | BC |
| 22D | 28 | 1, 8 | 5-480 | BC |
| 23D | 28 | 1, 8 | 40-200 | BC |
| 24D | 28 | 1, 8 | 80-160 | BC |
| 25D | 28 | 1, 8 | 90-130 | BC |
| 26D | 28 | 1, 8 | 140-240 | BC |
| 27D | 28 | 1, 8 | 160-220 | BC |
| 28D | 28 | 1, 8 | 180-200 | BC |
| 29D | 28 | 1, 8, 15 | 5-480 | BC |
| 30D | 28 | 1, 8, 15 | 40-200 | BC |
| 31D | 28 | 1, 8, 15 | 80-160 | BC |
| 32D | 28 | 1, 8, 15 | 90-130 | BC |
| 33D | 28 | 1, 8, 15 | 140-240 | BC |
| 34D | 28 | 1, 8, 15 | 160-220 | BC |
| 35D | 28 | 1, 8, 15 | 180-200 | BC |
| 36D | 21 | 1 | 5-480 | GC |
| 37D | 21 | 1 | 40-200 | GC |
| 38D | 21 | 1 | 80-160 | GC |
| 39D | 21 | 1 | 90-130 | GC |
| 40D | 21 | 1 | 140-240 | GC |
| 41D | 21 | 1 | 160-220 | GC |
| 42D | 21 | 1 | 180-200 | GC |
| 43D | 21 | 1, 8 | 5-480 | GC |
| 44D | 21 | 1, 8 | 40-200 | GC |
| 45D | 21 | 1, 8 | 80-160 | GC |
| 46D | 21 | 1, 8 | 90-130 | GC |
| 47D | 21 | 1, 8 | 140-240 | GC |
| 48D | 21 | 1, 8 | 160-220 | GC |
| 49D | 21 | 1, 8 | 180-200 | GC |
| 50D | 28 | 1 | 5-480 | GC |
| 51D | 28 | 1 | 40-200 | GC |
| 52D | 28 | 1 | 80-160 | GC |
| 53D | 28 | 1 | 90-130 | GC |
| 54D | 28 | 1 | 140-240 | GC |
| 55D | 28 | 1 | 160-220 | GC |
| 56D | 28 | 1 | 180-200 | GC |
| 57D | 28 | 1, 8 | 5-480 | GC |
| 58D | 28 | 1, 8 | 40-200 | GC |
| 59D | 28 | 1, 8 | 80-160 | GC |
| 60D | 28 | 1, 8 | 90-130 | GC |
| 61D | 28 | 1, 8 | 140-240 | GC |
| 62D | 28 | 1, 8 | 160-220 | GC |
| 63D | 28 | 1, 8 | 180-200 | GC |
| 64D | 28 | 1, 8, 15 | 5-480 | GC |
| 65D | 28 | 1, 8, 15 | 40-200 | GC |
| 66D | 28 | 1, 8, 15 | 80-160 | GC |
| 67D | 28 | 1, 8, 15 | 90-130 | GC |
| 68D | 28 | 1, 8, 15 | 140-240 | GC |
| 69D | 28 | 1, 8, 15 | 160-220 | GC |
| 70D | 28 | 1, 8, 15 | 180-200 | GC |
| 71D | 21 | 1 | 5-480 | CC |
| 72D | 21 | 1 | 40-200 | CC |
| 73D | 21 | 1 | 80-160 | CC |
| 74D | 21 | 1 | 90-130 | CC |
| 75D | 21 | 1 | 140-240 | CC |
| 76D | 21 | 1 | 160-220 | CC |
| 77D | 21 | 1 | 180-200 | CC |
| 78D | 21 | 1, 8 | 5-480 | CC |
| 79D | 21 | 1, 8 | 40-200 | CC |
| 80D | 21 | 1, 8 | 80-160 | CC |
| 81D | 21 | 1, 8 | 90-130 | CC |
| 82D | 21 | 1, 8 | 140-240 | CC |
| 83D | 21 | 1, 8 | 160-220 | CC |
| 84D | 21 | 1, 8 | 180-200 | CC |
| 85D | 28 | 1 | 5-480 | CC |
| 86D | 28 | 1 | 40-200 | CC |
| 87D | 28 | 1 | 80-160 | CC |
| 88D | 28 | 1 | 90-130 | CC |
| 89D | 28 | 1 | 140-240 | CC |
| 90D | 28 | 1 | 160-220 | CC |
| 91D | 28 | 1 | 180-200 | CC |
| 92D | 28 | 1, 8 | 5-480 | CC |
| 93D | 28 | 1, 8 | 40-200 | CC |
| 94D | 28 | 1, 8 | 80-160 | CC |
| 95D | 28 | 1, 8 | 90-130 | CC |
| 96D | 28 | 1, 8 | 140-240 | CC |
| 97D | 28 | 1, 8 | 160-220 | CC |
| 98D | 28 | 1, 8 | 180-200 | CC |
| 99D | 28 | 1, 8, 15 | 5-480 | CC |
| 100D | 28 | 1, 8, 15 | 40-200 | CC |
| 101D | 28 | 1, 8, 15 | 80-160 | CC |
| 102D | 28 | 1, 8, 15 | 90-130 | CC |
| 103D | 28 | 1, 8, 15 | 140-240 | CC |
| 104D | 28 | 1, 8, 15 | 160-220 | CC |
| 105D | 28 | 1, 8, 15 | 180-200 | CC |
| 106D | 21 | 1 | 5-480 | HC |
| 107D | 21 | 1 | 40-200 | HC |
| 108D | 21 | 1 | 80-160 | HC |
| 109D | 21 | 1 | 90-130 | HC |
| 110D | 21 | 1 | 140-240 | HC |
| 111D | 21 | 1 | 160-220 | HC |
| 112D | 21 | 1 | 180-200 | HC |
| 113D | 21 | 1, 8 | 5-480 | HC |
| 114D | 21 | 1, 8 | 40-200 | HC |
| 115D | 21 | 1, 8 | 80-160 | HC |
| 116D | 21 | 1, 8 | 90-130 | HC |
| 117D | 21 | 1, 8 | 140-240 | HC |
| 118D | 21 | 1, 8 | 160-220 | HC |
| 119D | 21 | 1, 8 | 180-200 | HC |
| 120D | 28 | 1 | 5-480 | HC |
| 121D | 28 | 1 | 40-200 | HC |
| 122D | 28 | 1 | 80-160 | HC |
| 123D | 28 | 1 | 90-130 | HC |
| 124D | 28 | 1 | 140-240 | HC |
| 125D | 28 | 1 | 160-220 | HC |
| 126D | 28 | 1 | 180-200 | HC |
| 127D | 28 | 1, 8 | 5-480 | HC |
| 128D | 28 | 1, 8 | 40-200 | HC |
| 129D | 28 | 1, 8 | 80-160 | HC |
| 130D | 28 | 1, 8 | 90-130 | HC |
| 131D | 28 | 1, 8 | 140-240 | HC |
| 132D | 28 | 1, 8 | 160-220 | HC |
| 133D | 28 | 1, 8 | 180-200 | HC |
| 134D | 28 | 1, 8, 15 | 5-480 | HC |
| 135D | 28 | 1, 8, 15 | 40-200 | HC |
| 136D | 28 | 1, 8, 15 | 80-160 | HC |
| 137D | 28 | 1, 8, 15 | 90-130 | HC |
| 138D | 28 | 1, 8, 15 | 140-240 | HC |
| 139D | 28 | 1, 8, 15 | 160-220 | HC |
| 140D | 28 | 1, 8, 15 | 180-200 | HC |
| 141D | 21 | 1 | 5-480 | EC |
| 142D | 21 | 1 | 40-200 | EC |
| 143D | 21 | 1 | 80-160 | EC |
| 144D | 21 | 1 | 90-130 | EC |
| 145D | 21 | 1 | 140-240 | EC |
| 146D | 21 | 1 | 160-220 | EC |
| 147D | 21 | 1 | 180-200 | EC |
| 148D | 21 | 1, 8 | 5-480 | EC |
| 149D | 21 | 1, 8 | 40-200 | EC |
| 150D | 21 | 1, 8 | 80-160 | EC |
| 151D | 21 | 1, 8 | 90-130 | EC |
| 152D | 21 | 1, 8 | 140-240 | EC |
| 153D | 21 | 1, 8 | 160-220 | EC |
| 154D | 21 | 1, 8 | 180-200 | EC |
| 155D | 28 | 1 | 5-480 | EC |
| 156D | 28 | 1 | 40-200 | EC |
| 157D | 28 | 1 | 80-160 | EC |
| 158D | 28 | 1 | 90-130 | EC |
| 159D | 28 | 1 | 140-240 | EC |
| 160D | 28 | 1 | 160-220 | EC |
| 161D | 28 | 1 | 180-200 | EC |
| 162D | 28 | 1, 8 | 5-480 | EC |
| 163D | 28 | 1, 8 | 40-200 | EC |
| 164D | 28 | 1, 8 | 80-160 | EC |
| 165D | 28 | 1, 8 | 90-130 | EC |
| 166D | 28 | 1, 8 | 140-240 | EC |
| 167D | 28 | 1, 8 | 160-220 | EC |
| 168D | 28 | 1, 8 | 180-200 | EC |
| 169D | 28 | 1, 8, 15 | 5-480 | EC |
| 170D | 28 | 1, 8, 15 | 40-200 | EC |
| 171D | 28 | 1, 8, 15 | 80-160 | EC |
| 172D | 28 | 1, 8, 15 | 90-130 | EC |
| 173D | 28 | 1, 8, 15 | 140-240 | EC |
| 174D | 28 | 1, 8, 15 | 160-220 | EC |
| 175D | 28 | 1, 8, 15 | 180-200 | EC |
| 176D | 21 | 1 | 5-480 | AML |
| 177D | 21 | 1 | 40-200 | AML |
| 178D | 21 | 1 | 80-160 | AML |
| 179D | 21 | 1 | 90-130 | AML |
| 180D | 21 | 1 | 140-240 | AML |
| 181D | 21 | 1 | 160-220 | AML |
| 182D | 21 | 1 | 180-200 | AML |
| 183D | 21 | 1, 8 | 5-480 | AML |
| 184D | 21 | 1, 8 | 40-200 | AML |
| 185D | 21 | 1, 8 | 80-160 | AML |
| 186D | 21 | 1, 8 | 90-130 | AML |
| 187D | 21 | 1, 8 | 140-240 | AML |
| 188D | 21 | 1, 8 | 160-220 | AML |
| 189D | 21 | 1, 8 | 180-200 | AML |
| 190D | 28 | 1 | 5-480 | AML |
| 191D | 28 | 1 | 40-200 | AML |
| 192D | 28 | 1 | 80-160 | AML |
| 193D | 28 | 1 | 90-130 | AML |
| 194D | 28 | 1 | 140-240 | AML |
| 195D | 28 | 1 | 160-220 | AML |
| 196D | 28 | 1 | 180-200 | AML |
| 197D | 28 | 1, 8 | 5-480 | AML |
| 198D | 28 | 1, 8 | 40-200 | AML |
| 199D | 28 | 1, 8 | 80-160 | AML |
| 200D | 28 | 1, 8 | 90-130 | AML |
| 201D | 28 | 1, 8 | 140-240 | AML |
| 202D | 28 | 1, 8 | 160-220 | AML |
| 203D | 28 | 1, 8 | 180-200 | AML |
| 204D | 28 | 1, 8, 15 | 5-480 | AML |
| 205D | 28 | 1, 8, 15 | 40-200 | AML |
| 206D | 28 | 1, 8, 15 | 80-160 | AML |
| 207D | 28 | 1, 8, 15 | 90-130 | AML |
| 208D | 28 | 1, 8, 15 | 140-240 | AML |
| 209D | 28 | 1, 8, 15 | 160-220 | AML |
| 210D | 28 | 1, 8, 15 | 180-200 | AML |

BC = Breast cancer (e.g. triple negative breast cancer or luminal B breast cancer, in particular triple negative breast cancer),
GC = gastric cancer,
CC = colorectal cancer,
HC = hepatocellular cancer,
EC = endometrial cancer,
AML = acute myeloid leukemia (e.g. Complex Karyotype AML).

As per the statement above, embodiment 1D refers to the situation wherein the neoplastic disease to be treated is breast cancer compound of formula (I) or a pharmaceutically acceptable salt thereof is administered to a patient according to a 21 day treatment cycle, wherein the compound of formula (I) or a pharmaceutically acceptable salt thereof is administered on day 1 of the treatment cycle and wherein the compound of formula (I) or a pharmaceutically acceptable salt thereof is administered to a patient at a dose corresponding to the mole equivalent of about 5 mg to about 480 mg of the free base of the compound of formula (I) per week in weeks when administered. The same applies analogously to embodiments 2D to 210D.

Generally, the compound of formula (I) or a pharmaceutically acceptable salt thereof will be administered at dosages that do not exceed the maximum tolerated dose (MTD) for a particular mode of administration and indication, as determined in a clinical dose escalation study.

### Formulations

The compound of formula (I) or a pharmaceutically acceptable salt thereof may be formulated as a pharmaceutical composition for non-parenteral administration, such as nasal, buccal, rectal, pulmonary, vaginal, sublingual, topical, transdermal, ophthalmic, otic or, especially, for oral administration, e.g. in the form of oral solid dosage forms, e.g. granules, pellets, powders, tablets, film or sugar coated tablets, effervescent tablets, hard and soft gelatin or HPMC capsules, coated as applicable, orally disintegrating tablets, oral solutions, lipid emulsions or suspensions, or for parenteral administration, such as intravenous, intramuscular, or subcutaneous, intrathecal, in-tradermal or epidural administration, to mammals, especially humans, e.g. in the form of solutions, lipid emulsions or suspensions containing microparticles or nanoparticles. The compositions may comprise the active ingredient(s) alone or, preferably, together with a pharmaceutically acceptable carrier.

The pharmaceutical compositions can be processed with pharmaceutically inert, inorganic or organic excipients for the production of oral solid dosage forms, e.g. granules, pellets, powders, tablets, film or sugar coated tablets, effervescent tablets, hard gelatin or HPMC capsules or orally disintegrating tablets. Fillers e.g. lactose, cellulose, mannitol, sorbitol, calcium phosphate, starch or derivatives thereof, binders e.g. cellulose, starch, polyvinylpyrrolidone, or derivatives thereof, glidants e.g. talcum, stearic acid or its salts, flowing agents e.g. fumed silica, can be used as such excipients for formulating and manufacturing of oral solid dosage forms, such as granules, pellets, powders, tablets, film or sugar coated tablets, effervescent tablets, hard gelatin or HPMC capsules, or orally disintegrating tablets. Suitable excipients for soft gelatin capsules are e.g. vegetable oils, waxes, fats, semisolid and liquid polyols etc.

Suitable excipients for the manufacture of oral solutions, lipid emulsions or suspensions are e.g. water, alcohols, polyols, saccharose, invert sugar, glucose etc. Suitable excipients for parenteral formulations are e.g. water, alcohols, polyols, glycerol, vegetable oils, lecithin, surfactants etc. Moreover, the pharmaceutical preparations can contain preservatives, solubilizers, stabilizers, wetting agents, emulsifiers, sweeteners, colorants, flavorants, salts for varying the osmotic pressure, buffers, masking agents or antioxidants. They can also contain other therapeutically valuable substances.

Pharmaceutical compositions suitable for injectable use include sterile aqueous solutions (where water soluble) or dispersions and sterile powders for the extem-porancous preparation of sterile injectable solutions or dispersion. For intravenous administration, suitable carriers include physiological saline, bacteriostatic water, Cremophor® EL or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. For intravenous injection of strongly lipophilic molecules it can be advantageous to include solubilizers in the formulation, for example surfactants, polymeric surfactants, polymers, complexing agents and/or co-solvents, which may significantly increase the solubility of the compounds in water. Examples of solubilizers include polyethylene glycol, propylene glycol, ethanol, glycerol and cyclodextrins (e.g. sulfobutyl ether-β-cyclodextrins).

In some embodiments the compound of formula (I) as the free base is provided as a pharmaceutical composition comprising a β-cyclodextrin e.g. for intravenous administration. The β-cyclodextrin may be sulfobutyl ether-β-cyclodextrin, e.g. CAS 182410-00-0, such as Captisol™ (Ligand) or Dexolve™ (Cyclolab).

Sterile injectable solutions can be prepared by incorporating the active compound in the required amount in an appropriate solvent with the compound of formula (I) or a pharmaceutically acceptable salt thereof as required, followed by filtered sterilization. Generally, dispersions are prepared by incorporating the active compound into a sterile vehicle that contains a dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In addition pharmaceutical compositions used in the invention optionally include buffers such as phosphate, citrate, or other organic acids; antioxidants including butylated hydroxytoluene (BHT), butylated hydroxyanisole (BHA), ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, or other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, the pharmaceutical compositions contain a pharmaceutically acceptable preservative. In some embodiments the preservative concentration ranges from 0.1 to 2.0 percent, typically v/v. Suitable preservatives include those known in the pharmaceutical arts, such as benzyl alcohol, phenol, m-cresol, methylparaben, and propy-lparaben.

In some embodiments the compound of formula (I) or a pharmaceutically acceptable salt thereof is formulated for intravenous administration with a suitable acceptable carrier.

The following numbered paragraphs describe particular embodiments of the invention.

Paragraph 1. A method for treating a neoplastic disease in a subject in need thereof, in particular a human, comprising administering a therapeutically effective amount of a compound of formula (I) or a pharmaceutically acceptable salt thereof to the subject according to an intermittent dosing schedule.

Paragraph 2. The method according to paragraph 1, wherein the compound of formula (I) or a pharmaceutically acceptable salt thereof is administered to the subject intravenously.

Paragraph 3. The method according to paragraph 1 or paragraph 2, wherein the dosing schedule comprises an interval of at least 2 days during which no compound of formula (I) or a pharmaceutically acceptable salt thereof is administered to the subject.

Paragraph 4. The method according to paragraph 1 or paragraph 2, wherein there is an interval of at least 7 days between each consecutive scheduled dose.

Paragraph 5. The method according to any one of paragraphs 1 to 4, wherein the compound of formula (I) or a pharmaceutically acceptable salt thereof is administered according to a three week treatment cycle, wherein the compound of formula (I) or a pharmaceutically acceptable salt thereof is administered in week one of the treatment cycle, e.g. on day 1, followed by two rest weeks.

Paragraph 6. The method according to any one of paragraphs 1 to 4, wherein the compound of formula (I) or a pharmaceutically acceptable salt thereof is administered according to a three week treatment cycle, wherein the compound of formula (I) or a pharmaceutically acceptable salt thereof is administered in week one and week two of the treatment cycle, e.g. on day 1 and day 8, followed by one rest week.

Paragraph 7. The method according to any one of paragraphs 1 to 4, wherein the compound of formula (I) or a pharmaceutically acceptable salt thereof is administered according to a four week treatment cycle, wherein the compound of formula (I) or a pharmaceutically acceptable salt thereof is administered in week one of the treatment cycle, e.g. on day 1, followed by three rest weeks.

Paragraph 8. The method according to any one of paragraphs 1 to 4, wherein the compound of formula (I) or a pharmaceutically acceptable salt thereof is administered according to a four week treatment cycle, wherein the compound of formula (I) or a pharmaceutically acceptable salt thereof is administered in week one and in week three of the treatment cycle,, e.g. on day 1 and day 15, with weeks two and four being rest weeks.

Paragraph 9. The method according to any one of paragraphs 1 to 4, wherein the compound of formula (I) or a pharmaceutically acceptable salt thereof is administered according to a four week treatment cycle, wherein the compound of formula (I) or a pharmaceutically acceptable salt thereof is administered in each week during the first three weeks of the treatment cycle, e.g. on day 1, day 8 and day 15, followed by a rest week.

Paragraph 10. The method according to any one of paragraphs 1 to 9, wherein the compound of formula (I) or a pharmaceutically acceptable salt thereof is administered to a patient at a dose corresponding to the mole equivalent of about 5 mg to about 480 mg of the free base of the compound of formula (I) per week during weeks when administered.

Paragraph 11. The method according to any one of paragraphs 1 to 9, wherein the compound of formula (I) or a pharmaceutically acceptable salt thereof is administered to a patient at a dose corresponding to the mole equivalent of about 40 mg to about 200 mg of the free base of the compound of formula (I) per week during weeks when administered.

Paragraph 12. The method according to any one of paragraphs 1 to 9, wherein the compound of formula (I) or a pharmaceutically acceptable salt thereof is administered to a patient at a dose corresponding to the mole equivalent of about 80 mg to about 160 mg of the free base of the compound of formula (I) per week during weeks when administered.

Paragraph 13. The method according to any one of paragraphs 1 to 9, wherein the compound of formula (I) or a pharmaceutically acceptable salt thereof is administered to a patient at a dose corresponding to the mole equivalent of about 90 mg to about 130 of the free base of the compound of formula (I) per week during weeks when administered.

Paragraph 14. The method according to any one of paragraphs 1 to 9, wherein the compound of formula (I) or a pharmaceutically acceptable salt thereof is administered to a patient at a dose corresponding to the mole equivalent of about 140 mg to about 240 mg of the free base of the compound of formula (I) per week during weeks when administered.

Paragraph 15. The method according to any one of paragraphs 1 to 9, wherein the compound of formula (I) or a pharmaceutically acceptable salt thereof is administered to a patient at a dose corresponding to the mole equivalent of about 160 mg to about 220 mg of the free base of the compound of formula (I) per week during weeks when administered.

Paragraph 16. The method according to any one of paragraphs 1 to 9, wherein the compound of formula (I) or a pharmaceutically acceptable salt thereof is administered to a patient at a dose corresponding to the mole equivalent of about 180 mg to about 200 mg of the free base of the compound of formula (I) per week during weeks when administered.

Paragraph 17. The method according to any one of paragraphs 1 to 16, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, naevi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, myeloproliferative disorders, lymphoproliferative disorders and myelodysplastic syndromes.

Paragraph 18. The method according to any one of paragraphs 1 to 17, wherein the neoplastic disease is one which is treatable by inhibition of PLK1 in addition to a treatment with a TTK inhibitor (e.g. the compound of formula (I)).

Paragraph 19. The method according to any one of paragraphs 1 to 19, wherein the neoplastic disease is a cancer, in particular a cancer selected from breast cancer (including triple negative breast cancer and luminal B breast cancer), gastric cancer, colorectal cancer, liver cancer (including hepatocellular cancer), endometrial cancer, ovarian cancer, esophageal cancer, lung cancer (including non-small cell lung cancer), Kaposi's sarcoma, cervical cancer, pancreatic cancer, melanoma, prostate cancer, bladder cancer and leukemia, e.g. acute myeloid leukemia (AML) (including Complex Karyotype AML).

Paragraph 20. The method according to any one of paragraphs 1 to 19, wherein the neoplastic disease is breast cancer.

Paragraph 21. The method according to any one of paragraphs 1 to 19, wherein the neoplastic disease is triple negative breast cancer.

Paragraph 22. The method according to any one of paragraphs 1 to 19, wherein the neoplastic disease is luminal B breast cancer.

Paragraph 23. The method according to any one of paragraphs 1 to 19, wherein the neoplastic disease is gastric cancer.

Paragraph 24. The method according to any one of paragraphs 1 to 19, wherein the neoplastic disease is colorectal cancer.

Paragraph 25. The method according to any one of paragraphs 1 to 19, wherein the neoplastic disease is hepatocellular cancer.

Paragraph 26. The method according to any one of paragraphs 1 to 19, wherein the neoplastic disease is endometrial cancer.

Paragraph 27. The method according to any one of paragraphs 1 to 19, wherein the neoplastic disease is acute myeloid leukemia (AML)

Paragraph 28. The method according to any one of paragraphs 1 to 19, wherein the neoplastic disease is Complex Karyotype AML.

Paragraph 1A. A compound of formula (I) or a pharmaceutically acceptable salt thereof for use in the treatment of a neoplastic disease in a subject, in particular a human, wherein the treatment comprises administering the compound of formula (I) or a pharmaceutically acceptable salt thereof to the subject according to an intermittent dosing schedule.

Paragraph 2A. The compound for use according to paragraph 1A, wherein the compound of formula (I) or a pharmaceutically acceptable salt thereof is administered to the subject intravenously.

Paragraph 3A. The compound for use according to paragraph 1A or paragraph 2A, wherein the dosing schedule comprises an interval of at least 2 days during which no compound of formula (I) or a pharmaceutically acceptable salt thereof is administered to the subject.

Paragraph 4A. The compound for use according to paragraph 1A or paragraph 2A, wherein there is an interval of at least 7 days between each consecutive scheduled dose.

Paragraph 5A. The compound for use according to any one of paragraphs 1A to 4A, wherein the compound of formula (I) or a pharmaceutically acceptable salt thereof is administered according to a three week treatment cycle, wherein the compound of formula (I) or a pharmaceutically acceptable salt thereof is administered in week one of the treatment cycle, e.g. on day 1, followed by two rest weeks.

Paragraph 6A. The compound for use according to any one of paragraphs 1A to 4A, wherein the compound of formula (I) or a pharmaceutically acceptable salt thereof is administered according to a three week treatment cycle, wherein the compound of formula (I) or a pharmaceutically acceptable salt thereof is administered in week one and week two of the treatment cycle, e.g. on day 1 and day 8, followed by one rest week.

Paragraph 7A. The compound for use according to any one of paragraphs 1A to 4A, wherein the compound of formula (I) or a pharmaceutically acceptable salt thereof is administered according to a four week treatment cycle, wherein the compound of formula (I) or a pharmaceutically acceptable salt thereof is administered in week one of the treatment cycle, e.g. on day 1, followed by three rest weeks.

Paragraph 8A. The compound for use according to any one of paragraphs 1A to 4A, wherein the compound of formula (I) or a pharmaceutically acceptable salt thereof is administered according to a four week treatment cycle, wherein the compound of formula (I) or a pharmaceutically acceptable salt thereof is administered in week one and in week three of the treatment cycle,, e.g. on day 1 and day 15, with weeks two and four being rest weeks.

Paragraph 9A. The compound for use according to any one of paragraphs 1A to 4A, wherein the compound of formula (I) or a pharmaceutically acceptable salt thereof is administered according to a four week treatment cycle, wherein the compound of formula (I) or a pharmaceutically acceptable salt thereof is administered in each week during the first three weeks of the treatment cycle, e.g. on day 1, day 8 and day 15, followed by a rest week.

Paragraph 10A. The compound for use according to any one of paragraphs 1A to 9A, wherein the compound of formula (I) or a pharmaceutically acceptable salt thereof is administered to a patient at a dose corresponding to the mole equivalent of about 5 mg to about 480 mg of the free base of the compound of formula (I) per week during weeks when administered.

Paragraph 11A. The compound for use according to any one of paragraphs 1A to 9A, wherein the compound of formula (I) or a pharmaceutically acceptable salt thereof is administered to a patient at a dose corresponding to the mole equivalent of about 40 mg to about 200 mg of the free base of the compound of formula (I) per week during weeks when administered.

Paragraph 12A. The compound for use according to any one of paragraphs 1A to 9A, wherein the compound of formula (I) or a pharmaceutically acceptable salt thereof is administered to a patient at a dose corresponding to the mole equivalent of about 80 mg to about 160 mg of the free base of the compound of formula (I) per week during weeks when administered.

Paragraph 13A. The compound for use according to any one of paragraphs 1A to 9A, wherein the compound of formula (I) or a pharmaceutically acceptable salt thereof is administered to a patient at a dose corresponding to the mole equivalent of about 90 mg to about 130 of the free base of the compound of formula (I) per week during weeks when administered.

Paragraph 14A. The compound for use according to any one of paragraphs 1A to 9A, wherein the compound of formula (I) or a pharmaceutically acceptable salt thereof is administered to a patient at a dose corresponding to the mole equivalent of about 140 mg to about 240 mg of the free base of the compound of formula (I) per week during weeks when administered.

Paragraph 15A. The compound for use according to any one of paragraphs 1A to 9A, wherein the compound of formula (I) or a pharmaceutically acceptable salt thereof is administered to a patient at a dose corresponding to the mole equivalent of about 160 mg to about 220 mg of the free base of the compound of formula (I) per week during weeks when administered.

Paragraph 16A. The compound for use according to any one of paragraphs 1A to 9A, wherein the compound of formula (I) or a pharmaceutically acceptable salt thereof is administered to a patient at a dose corresponding to the mole equivalent of about 180 mg to about 200 mg of the free base of the compound of formula (I) per week during weeks when administered.

Paragraph 17A. The compound for use according to any one of paragraphs 1A to 16A, wherein the neoplastic disease is a solid tumor.

Paragraph 18A. The compound for use according to any one of paragraphs 1A to 17A, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, nacvi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neurocpitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, myeloproliferative disorders, lymphoproliferative disorders and myelodysplastic syndromes.

Paragraph 19A. The compound for use according to any one of paragraphs 1A to 18A, wherein the neoplastic disease is one which is treatable by inhibition of PLK1 in addition to a treatment with a TTK inhibitor (e.g. the compound of formula (I)).

Paragraph 20A. The compound for use according to any one of paragraphs 1A to 19A, wherein the neoplastic disease is a cancer, in particular a cancer selected from breast cancer (including triple negative breast cancer and luminal B breast cancer), gastric cancer, colorectal cancer, liver cancer (including hepatocellular cancer), endometrial cancer, ovarian cancer, esophageal cancer, lung cancer (including non-small cell lung cancer), Kaposi's sarcoma, cervical cancer, pancreatic cancer, melanoma, prostate cancer, bladder cancer and leukemia, e.g. acute myeloid leukemia (AML) (including Complex Karyotype AML).

Paragraph 21A. The compound for use according to any one of paragraphs 1A to 19A, wherein the neoplastic disease is breast cancer.

Paragraph 22A. The compound for use according to any one of paragraphs 1A to 19A, wherein the neoplastic disease is triple negative breast cancer.

Paragraph 23A. The compound for use according to any one of paragraphs 1A to 19A, wherein the neoplastic disease is luminal B breast cancer.

Paragraph 24A. The compound for use according to any one of paragraphs 1A to 19A, wherein the neoplastic disease is gastric cancer.

Paragraph 25A. The compound for use according to any one of paragraphs 1A to 19A, wherein the neoplastic disease is colorectal cancer.

Paragraph 26A. The compound for use according to any one of paragraphs 1A to 19A, wherein the neoplastic disease is hepatocellular cancer.

Paragraph 27A. The compound for use according to any one of paragraphs 1A to 19A, wherein the neoplastic disease is endometrial cancer.

Paragraph 28A. The compound for use according to any one of paragraphs 1A to 19A, wherein the neoplastic disease is acute myeloid leukemia (AML)

Paragraph 29A. The compound for use according to any one of paragraphs 1A to 19A, wherein the neoplastic disease is Complex Karyotype AML.

Paragraph 1B. Use of a compound of formula (I) or a pharmaceutically acceptable salt thereof in the preparation of a medicament for the treatment of a neoplastic disease in a subject, wherein said treatment comprises administering a compound of formula (I) or a pharmaceutically acceptable salt thereof to the subject according to an in-termittent dosing schedule.

Paragraph 2B. The use according to paragraph 1B, wherein the compound of formula (I) or a pharmaceutically acceptable salt thereof is administered to the subject as defined in any one of paragraphs 2A to 16A.

Paragraph 3B. The use according to paragraph 1B or paragraph 2B, wherein the neoplastic disease is as defined in any one of paragraphs 17A to 29A.

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Particular embodiments of the invention are described in the following Examples, which serve to illustrate the invention in more detail and should not be construed as limiting the invention in any way.

### EXAMPLES

Methodology Kinase Assay

A radiometric protein kinase assay (33PanQinase® Activity Assay) was used for measuring the kinase activity of TTK and PLK1. TTK and PLKI protein kinases were expressed as recombinant full-length GST-fusion proteins. The reaction cocktail contained 25 mL of assay buffer (standard buffer/[Y-33P]-ATP) and 10 mL of ATP solution (in water), 5 mL of test compound and 10 mL of enzyme/substrate mixture. The assay for the protein kinases contained 70 mM HEPES-NaOH pH 7.5, 3 mM MgCl2, 3 mM MnC12, 3 mM Na-orthovanadate, 1.2 mM DTT, 50 mg/ml PEG20000, ATP (0.3 mM for TTK and 1 mM for PLK1), [Y-33P]-ATP (approx. 8×105 cpm per well), protein kinase (15.8 nM for TTK and 5 nM for PLK1), and substrate (1 mg/50 mL for TTK and 2 mg/50 mL for PLK1). All assays were performed with a Beck-manCoulter/SAGIAN™ Core System. The fitting model for the IC50 determinations was “Sigmoidal response (variable slope)” with parameters “top” fixed at 100% and “bottom” at 0%. The fitting method used was a least squares fit.

### Target Residence Time Assays

The affinity, i.e. the equilibrium dissociation constant (KD) (referred to as residence times), of the compound of formula (I) against TTK and PLK1 were determined using a Biacore T200TM surface plasmon resonance instrument using recombinant expressed TTK kinase domain (amino acids 519-808) or biotinylated PLK1. The immobilization of TTK was performed as described in Maia et al. Annals of Oncology, 2015; 26:2180-2192. The immobilization of biotinylated PLKI was performed as described in Willemsen-Seegers et al. Journal of Molecular Biology, 2017; 429:574-586. The subsequent single cycle kinetic assays were performed at 22° C. using a compound concentration gradient of 1, 3.6, 10, 31.6 and 100 nM for TTK and 10, 31.6, 100, 316 and 1000 nM for PLK1, a contact time of 100 s and a flow rate of 30 mL/min. The dissociation period was 1200-1800 s and a correction for an unstable surface using a blank run with buffer was performed. Binding kinetics were calculated based on the binding curves, demonstrating good signal-to-noise ratios for all compounds tested (data not shown).

### Xenograft Studies

Female NCr nu/nu nude mice (CharlesRiver laboratories) of 8-12 weeks of age were subcutaneously (s.c.) inoculated in the flank with 5×106 MDA-MB-231 tumor cells. Randomization into treatment groups (8 mice per group) was made when the mean tumor size was 100-150 mm3. Mice were treated with the different compounds and schedules as indicated in the Figures.

Body weights and tumor volumes were determined at least twice per week by measuring two dimensions with calipers and applying the formula “V=(L×W2)/2”, where V is the tumor volume, and L and W are the tumor length and width respectively. Individual mice were culled when tumors reached 1500 mm3 or more, or when the body weight loss (BWL) was found to have exceeded 20%. Mice were also culled if the BWL was determined to be >15% for 3 consecutive days. Any mice with >10% BWL received a dosing holiday until the BWL returned to <10%. All animal protocols were reviewed and approved by the relevant local committees in USA (IACUC) where the studies were performed.

Pharmacokinetic (PK) studies

In a first study groups 1, 2, and 3 (9 mice each) received a single oral dose administration of 5, 7.5, or 10 mg/kg in formulation 1, respectively. Groups 4, 5, and 6 (9 mice each) received a single oral dose administration of 5, 7.5, or 10 mg/kg in formulation 2, respectively. Plasma samples were collected on day 1 for Groups 1 to 6. Formulation 1 consisted of DMSO, cremophor EL, and 5% mannitol in water (10/10/80; v/v/v). Formulation 2 consisted of ethanol, PEG 400, and citric acid 20 mM (10/10/80; v/v/v). Blood samples were collected at 1, 2, 4, 8, 12, and 24 h post-administration (3 mice/time points).

In a second study female Swiss Albino mice were administered daily oral dose of 5, 7.5, or 10 mg/kg of the compound of formula (I) for 5 days. Groups 1, 2, and 3 (9 mice each) received a daily oral dose of 5, 7.5, or 10 mg/kg for 5 days in formulation 1, respectively. Groups 4, 5, and 6 (9 mice each) received a daily oral dose of 5, 7.5, or 10 mg/kg for 5 days in formulation 2, respectively. PK samples were collected on day 5 for groups 1 to 6. Formulation 1 consisted of DMSO, cremophor EL, and 5% mannitol in water (10/10/80; v/v/v). Formulation 2 consisted of ethanol, PEG 400, and citric acid 20 mM (10/10/80; v/v/v). Blood samples were collected at 1, 2, 4, 8, 12, and 24 h post-administration (3 mice/time points).

In a third study female Swiss Albino mice were administered intravenously (bolus, 5 mL/kg) dose of 5 mg/kg of the compound of formula (I). Group 1 (9 mice) received a single i.v. dose administration on day 1. Group 2 (9 mice) received daily oral administration of 10 mg/kg of the compound of formula (I) from day 1 to day 5 following by a single i.v. dose administration of 5 mg/kg of the compound of formula (I) on day 6. PK samples were collected on day 1 for group 1 and on day 6 for group 2. The formulation vehicle consisted of ethanol, PEG 400 and citric acid 20 mM (10/10/80; v/v/v) in water for injection (WFI). Blood samples were taken pre-dose and at 0.083, 0.25, 0.5, 1, 2, 6, 12, and 24 h post-dose administration (3 mice/time point).

In each of the three studies blood was collected via the sephanous vein into K2-EDTA tubes kept on ice until centrifugation at 4° C. Plasma was stored at ap-proximately-80° C. Sample work-up for analytics consisted of 15 μL plasma mixed with 45 μL acetonitrile containing the compound of formula (I)-d3 as internal standard, followed by centrifugation and injection of 2 μL of the supernatant into the LC-MS/MS system. PK parameters were calculated using Phoenix® WinNonLin 6.4. PK analysis was based on sparse sampling.

### Results

TTK and PLK1 are kinases with an essential role in control of the spindle assembly checkpoint (SAC), which is a cell cycle surveillance mechanism ensuring optimal cell division via proper chromosome alignment. TTK and PLKI co-operate to recruit SAC components to the SAC protein complex at the kinetochore of the chromosome, thus inhibition of both enzymes should maximize progression of mitosis via more rapid breakage of the SAC (Von Schubert et al., Cell Reports 2015, 12;66-78). This has proven to be the case in tumor cell systems when comparing the compound of formula (I) with TKK inhibitors not having any meaningful PLKI inhibitory activity (data not shown).

In general, the compound of formula (I) shows strong specificity for TTK, with other kinase IC50s being at least 10-fold higher than those for TTK. The TTK kinase assay described above confirmed that the compound of formula (I) is highly potent against TTK, giving an IC50 of 7 nM (0.4 nM when measured as described in WO 2015/155042).

The PLK1 kinase assay described above also showed that the compound of formula (I) targets PLK1. The compound of formula (I) was found to inhibit PLK1 with an

IC50 of 72 nM. When measured as described in WO 2015/155042 the compound of formula (I) inhibited PLK1 with an IC50 of 46 nM. Other TTK inhibitors reported in the literature have similar or slightly better overall specificity for TTK but conversely have little or no activity against PLKI relative to their activity against TTK (data not shown). Importantly, the compound of formula (I) has a very long target-residency of >12 h on TTK, while that for PLKI is just a few minutes. This prolonged inhibition of TTK combined with a transient effect on PLKI leads to a rapid disruption of the SAC leaving the cells without adequate time for correct chromosome segregation.

Prolonged TTK target occupancy was also measured in tumors derived from MDA-MB-231 xenograft-bearing mice treated with intermittent i.v. dosing regimen of compound of formula (I). To determine the tumor TTK target occupancy time of the compound of formula (I), mice harboring the TNBC xenograft model MDA-MB-231 were treated IV, twice-weekly with MTD and sub-MTD doses of the compound of formula (I). Analysis of vehicle-and compound of formula (I)-treated tumors for TTK target occupancy indicated that the compound of formula (I) occupied tumor-derived TTK in a concentration-dependent manner; TTK was completely occupied by the compound of formula (I) for at least 72 h after administration of the last MTD dose. A repeat experiment using IV, weekly MTD dosing indicated that tumor-derived TTK was fully drug-occupied for up to 6 days after the last administration.

In cellular systems, the compound of formula (I) has high potency against sensitive cells. In a 5-day anti-proliferative screen of 18 different triple negative breast cancer cell lines (TNBC), the compound of formula (I) had a median GI50 of 35 nM. In mice, the compound of formula (I) has shown significant activity against tumor patient-derived xenografts (PDX), including TNBC and hepatocellular cancer (HCC) models, with effects ranging from minimal to very strong including substantial regression (data not shown).

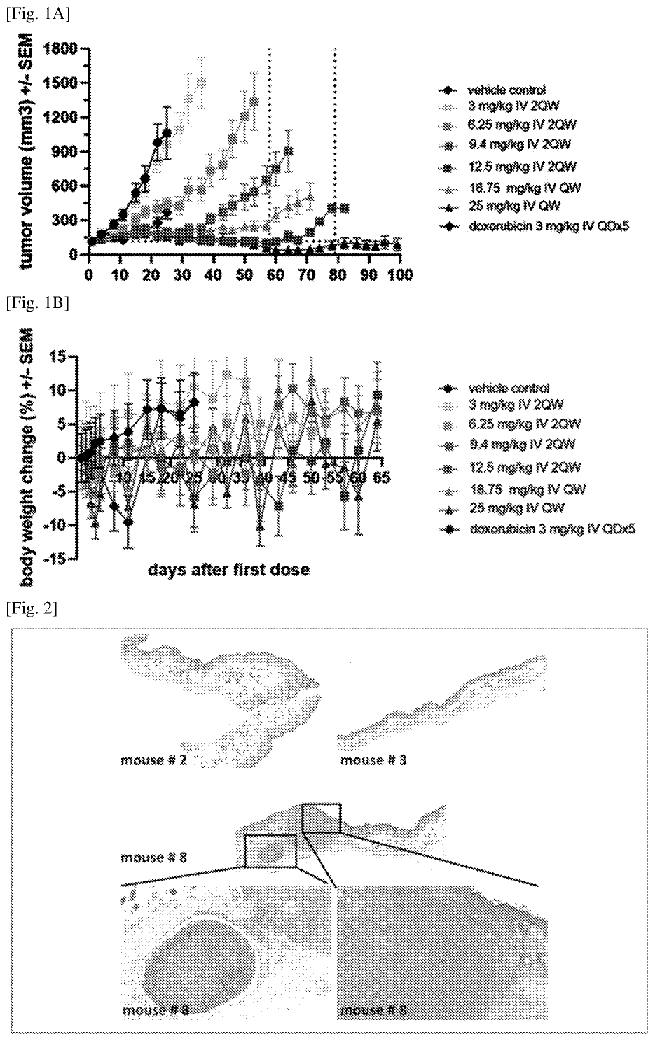

Efficacy of the compound of formula (I) was extensively tested in the TNBC xenograft model MDA-MB-231, in which weckly (QW) and twice-weekly (2QW) in-termittent i.v. dosing schedules were evaluated (FIG. 1). For both schedules, the compound of formula (I) was administered at the MTD and fractions thereof (QW dosing at MTD: 25 mg/kg and 0.75 ×MTD: 18.75 mg/kg; 2QW dosing at MTD: 12.5 mg/kg, 0.75×MTD: 9.4 mg/kg, 0.5×MTD: 6.25 mg/kg, and 0.25×MTD: 3 mg/kg).

All treatments were in general well tolerated as judged by changes in body weight with no drug-related animal deaths recorded. Dosing of the compound of formula (I) at the MTD had some influence on body weight decreases, although body weight loss remained within an acceptable 10% threshold. With MTD-dosing, tumor stasis and tumor regressions were observed. Weekly IV-dosing was associated with the most potent anti-tumor activity. While the different fractions of the MTD dosing for both QW and 2QW showed dose-dependent anti-tumor activity, generally QW dosing showed higher activity, despite the total weekly dose administered being identical between both schedules.

The mice from the weekly MTD dosing group were observed for tumor regrowth for an additional 20 days after treatment cessation on D100. Strikingly, three of eight tumors continued to shrink to a volume≤6 mm3, and were therefore investigated for the presence of residual tumor cells by histopathological analysis of the tumor implantation site and the surrounding tissue/skin. Two mice showed no detectable residual tumor cells, while one mouse had two small aggregates of neoplastic cells at the inoculation site (FIG. 2). Accordingly, 25% of the mice treated with weekly with the compound of formula (I) at the MTD-dose could be considered as cured based on pathological analysis (Table 1).

TABLE 1
Summary of pathological evaluation for cures

| Dose of Formula (I) | Mouse number | Tumor-free | Tumor-free (%) |
|---|---|---|---|
| 25 mg/kg, QW, IV | 2 and 3 | Yes | 25 |
| | 8 | No | |

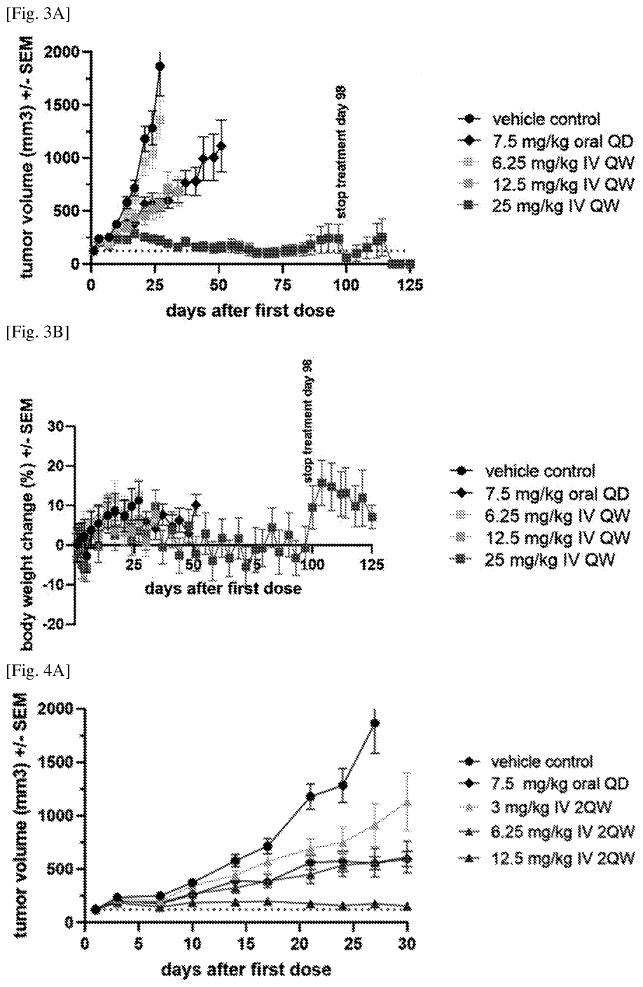

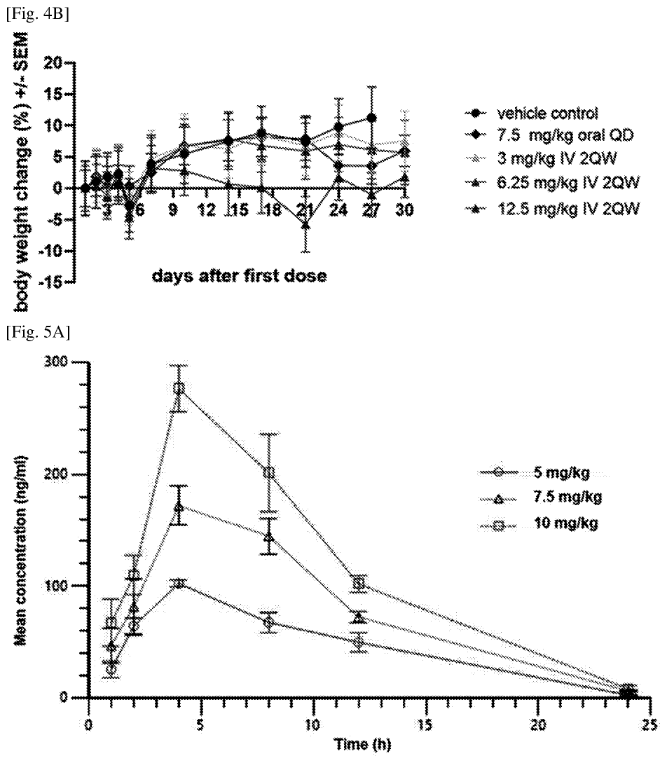

Since the compound of formula (I) can also be administered orally, it was of interest to compare efficacy after daily oral administration to that obtained with intermittent i.v. dosing in the same tumor model. Intermittent i.v. dosing was performed with doses at the MTD, 0.5×MTD, and 0.25×MTD, with both QW and 2QW regimens (see legend of graphs for exact doses), while daily oral dosing was performed at the MTD only. A dose-dependent efficacy was observed for both i.v. dosing schedules, with MTD i.v. dosing being far superior to MTD oral daily treatment, independent of the schedule; 0.5×MTD i.v. dosing elicited equipotent efficacy as MTD daily oral dosing (see FIG. 3 and FIG. 4).

All treatments were relatively well tolerated, with no animal deaths. For 2QW MTD dosing, treatment was stopped at Day-30 due to tail vein irritations (scabbing) induced by the repeated IV-injections. In contrast, QW MTD dosing was continued until Day-98, with a potent maintenance of anti-tumor activity. Subsequently, the animals were observed for tumor regrowth until Day-125. Consistent with the data above, two of eight animals (25%) showed no measurable/palpable tumor mass from Day-80 to Day-125; the tumor implantation site in these two animals, including surrounding tissue and skin, was therefore resected and processed for FFPE, followed by H&E staining and investigation for residual tumor cells. Both animals were consequently defined as tumor-free.

Taken together, the data obtained from the MDA-MB-231 TNBC tumor model suggest that intermittent i.v. administration can achieve a higher anti-cancer potency than daily oral dosing, with tumor regressions and pathologically-confirmed cures observed.

The first PK study compared the exposures of the compound of formula (I) in female Swiss Albino mice after single oral administration at 3 different dose levels and using two different formulations. The derived PK parameters are presented in Table 2 and the mean concentration-time profiles are depicted in FIG. 5. After oral administration, the compound of formula (I) was slowly absorbed with a median Tmax of 4 h. The maximum plasma concentration (Cmax) and the systemic exposure (AUClast) of the compound of formula (I) increased in a roughly dose-proportional manner between 5 and 10 mg/kg independently of the formulations used. The exposures observed using formulation 2 (citric acid) showed a slightly higher exposure than formulation 1. The oral bioavailability after single dose administration was high with a range of 76% to 96% based on exposure observed after i.v. administration.

TABLE 2
Mean (±SD) derived plasma PK parameters after single oral dose
administration of compound of formula (I) in female mice

| | Formulation 1 | | | Formulation 2 | | |
|---|---|---|---|---|---|---|
| Dose (mg/kg) | 5 | 7.5 | 10 | 5 | 7.5 | 10 |
| Tmax (h) | 4 | 4 | 4 | 4 | 4 | 4 |
| Cmax (ng/mL) | 108 (11.6) | 164 (20.8) | 210 (45.6) | 103 (4.41) | 172 (31.1) | 276 (35.0) |
| Cmax/Dose | 21.6 | 21.9 | 21 | 20.6 | 22.9 | 27.6 |
| ((ng/mL)/(mg/kg)) | | | | | | |
| Apparent T1/2 (h) | 3.27 | 3.23 | 3.66 | 3.29 | 3.51 | 3.43 |
| AUClast (h*ng/mL) | 1080 | 1650 | 2430 | 1120 | 1880 | 2730 |
| AUClast/Dose | 216 | 220 | 243 | 224 | 251 | 273 |
| ((h*ng/mL)/(mg/kg)) | | | | | | |
| AUCinf (h*ng/mL) | 1100 | 1660 | 2480 | 1130 | 1910 | 2770 |
| AUCinf/Dose | 220 | 221 | 248 | 226 | 255 | 277 |
| (h*ng/mL/mg/kg) | | | | | | |
| % extrapolated | 1.11 | 1.06 | 1.9 | 1.14 | 1.67 | 1.47 |
| F (%)* | 76 | 77 | 86 | 78 | 88 | 96 |

AUClast corresponds to AUC0-24 h
*Based on exposure observed after i.v. administration

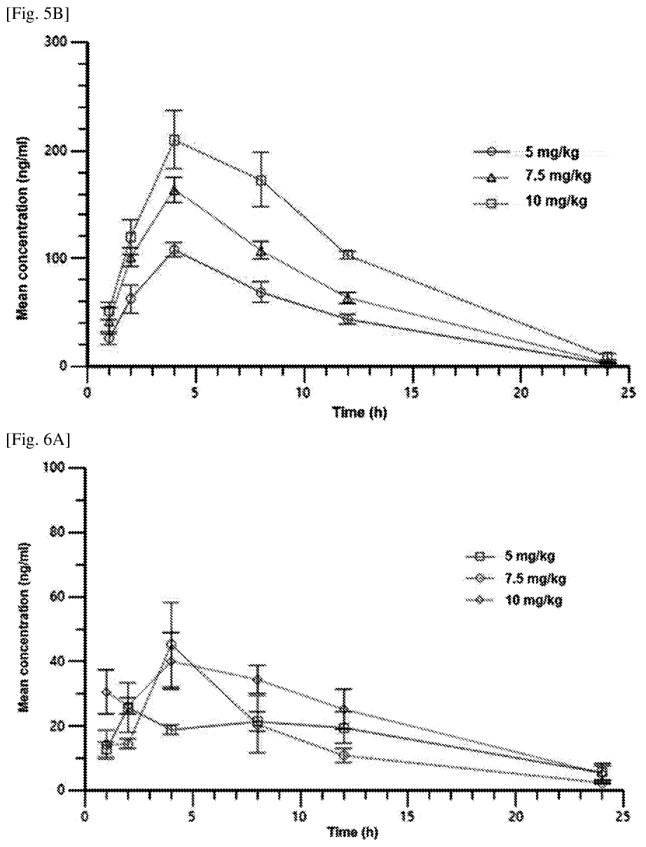

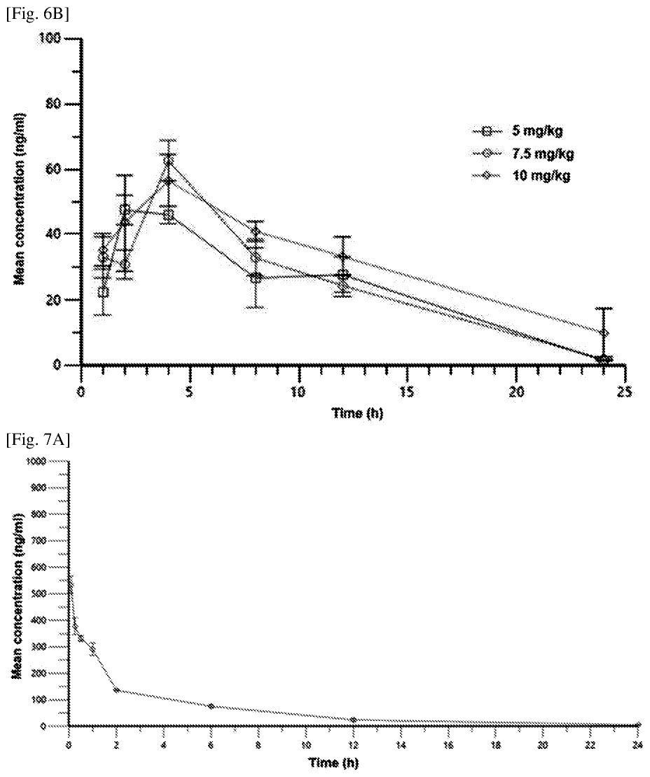

The second PK study compared the exposures of the compound of formula (I) in female Swiss Albino mice on day 5 after daily oral administration using two different formulations. The derived PK parameters are presented in Table 3 and mean con-centration-time profiles are depicted in FIG. 6. After oral administration, the compound of formula (I) was slowly absorbed with a median Tmax between 2 h and 4 h. The maximum concentration (Cmax) and the systemic exposure (AUClast) of the compound of formula (I) after multiple daily oral administration increased in a less than dose-proportional manner between 5 and 10 mg/kg independently of the formulations used. Exposures were higher with formulation 2 (citric acid). The oral bioavailability after multiple daily dose administration was low to moderate with a range of 18% to 41% (Table 3) based on exposure observed after i.v. administration.

TABLE 3
Mean derived plasma PK parameters on day 5 after daily oral dose
administration of the compound of formula (I) in female mice

| | Formulation 1 | | | Formulation 2 | | |
|---|---|---|---|---|---|---|
| Dose (mg/kg) | 5 | 7.5 | 10 | 5 | 7.5 | 10 |
| Tmax (h) | 2 | 4 | 4 | 2 | 4 | 4 |
| Cmax (ng/mL) | 25.8 | 45.2 | 40.1 | 47.4 | 62.7 | 56.5 |
| | (13.5) | (22.5) | (15.0) | (7.72) | (10.5) | (13.9) |
| Cmax/Dose | 5.16 | 6.03 | 4.01 | 9.48 | 8.36 | 5.65 |
| ((ng/mL)/(mg/kg)) | | | | | | |
| Apparent T1/2 (h) | 7.8 | 5.31 | 5.81 | 4.02 | 3.79 | 7.66 |
| AUClast (h*ng/mL) | 374 | 356 | 563 | 570 | 607 | 760 |
| AUClast/Dose | 74.8 | 47.5 | 56.3 | 114 | 80.9 | 76 |
| ((h*ng/mL)/(mg/kg)) | | | | | | |
| AUCinf (h*ng/mL) | 408 | 375 | 608 | 579 | 618 | 870 |
| AUCinf/Dose | 81.6 | 50 | 60.8 | 116 | 82.4 | 87 |
| (h*ng/mL/mg/kg) | | | | | | |
| % extrapolated | 8.33 | 5.04 | 7.41 | 1.53 | 1.77 | 12.7 |
| F (%)* | 29 | 18 | 22 | 41 | 29 | 31 |

AUClast corresponds to AUC0-24 h
*Based on exposure observed after iv administration

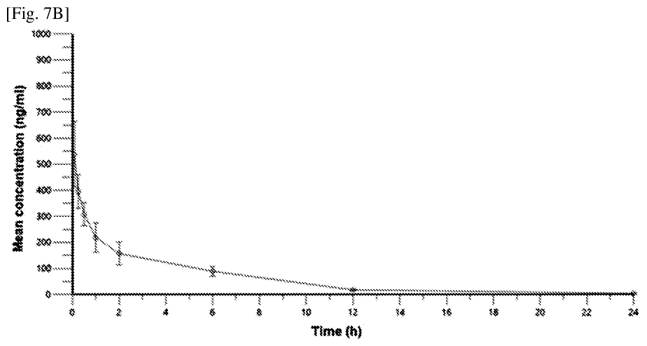

The third PK study compared the exposures of the compound of formula (I) in female Swiss Albino mice after 5 mg/kg intravenous administration on day 1 and on day 6 (on day 6 following daily oral administration of 10 mg/kg the compound of formula (I)). The derived PK parameters are presented in Table 4 and mean con-centration-time profiles are depicted in FIG. 7. After intravenous administration, the PK parameters showed no apparent difference between group 1 and group 2 (Table 4) whereas a decrease in exposures was observed after daily oral administration of compound of formula (I). A large volume of distribution (greater than total body water; 15.4 to 19.0 L/kg) and a moderate plasma clearance (55.4 to 57.9 mL/min/kg) were observed resulting in a half-life of 4.00 to 4.70 h for both groups. The concentrations at 24 hours were more than 100-fold lower than the CO, suggesting that AUCO-24h properly reflected the total exposure. AUCinf was calculated with confidence with a maximum of 2.54% extrapolation. Exposures following intravenous administration on day 1 and on day 6 (on day 6 following daily oral dose administration) in term of AUCO-24h was similar between day 1 and day 6. The data suggest that the daily administration of oral dose of 10 mg/kg the compound of formula (I) does not induce systemic induction.

TABLE 4
Mean derived plasma PK parameters after single iv administration (group 1)
and after multiple oral administration for 5 days followed by a single
intravenous administration on day 6 (group 2) of the compound
of formula (I) in female mice

| | Group 1, Day 1 | Group 2, Day 6 |
|---|---|---|
| C0 (ng/mL) | 636 | 627 |
| Clast (ng/mL) | 5.4 | 4.29 |
| AUClast (h*ng/mL) | 1400 | 1480 |
| AUCinf (h*ng/mL) | 1440 | 1500 |
| AUC_% extrapolated | 2.54 | 1.65 |
| T1/2 (h) | 4.7 | 4 |
| CL (mL/min/kg) | 57.9 | 55.4 |
| Vss (L/kg) | 19 | 15.4 |

AUClast corresponds to ALC0-24 h

1. A method for the treatment of a neoplastic disease in a subject, wherein the treatment comprises:
administering the compound of formula (I) 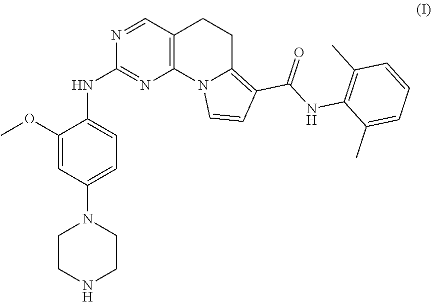 [SMILES: COc1cc(N2CCNCC2)ccc1Nc1ncc2c(n1)-n1ccc(C(=O)Nc3c(C)cccc3C)c1CC2]
or a pharmaceutically acceptable salt thereof to the subject according to an intermittent dosing schedule.

2. The method according to claim 1, wherein the compound of formula (I) or a pharmaceutically acceptable salt thereof is administered to the subject intravenously.

3. The method according to claim 1, wherein the dosing schedule comprises an interval of at least 2 days during which no compound of formula (I) or a pharmaceutically acceptable salt thereof is administered to the subject.

4. The method according to claim 1, wherein there is an interval of at least 7 days between each consecutive scheduled dose.

5. The method according to claim 1, wherein the compound of formula (I) or a pharmaceutically acceptable salt thereof is administered according to a three week treatment cycle, and wherein the compound of formula (I) or a pharmaceutically acceptable salt thereof is administered in week one of the treatment cycle followed by two rest weeks.

6. The method according to claim 1, wherein the compound of formula (I) or a pharmaceutically acceptable salt thereof is administered according to a three week treatment cycle, and wherein the compound of formula (I) or a pharmaceutically acceptable salt thereof is administered in week one and week two of the treatment cycle followed by one rest week.

7. The method according to claim 1, wherein the compound of formula (I) or a pharmaceutically acceptable salt thereof is administered according to a four week treatment cycle, and wherein the compound of formula (I) or a pharmaceutically acceptable salt thereof is administered in week one of the treatment cycle followed by three rest weeks.

8. The method according to claim 1, wherein the compound of formula (I) or a pharmaceutically acceptable salt thereof is administered according to a four week treatment cycle, and wherein the compound of formula (I) or a pharmaceutically acceptable salt thereof is administered in week one and in week three of the treatment cycle, with weeks two and four being rest weeks.

9. The method according to claim 1, wherein the compound of formula (I) or a pharmaceutically acceptable salt thereof is administered according to a four week treatment cycle, and wherein the compound of formula (I) or a pharmaceutically acceptable salt thereof is administered in each week during the first three weeks of the treatment cycle followed by a rest week.

10. The method according to claim 1, wherein the compound of formula (I) or a pharmaceutically acceptable salt thereof is administered to a patient at a dose corresponding to the mole equivalent of about 40 mg to about 200 mg of the free base of the compound of formula (I) per week during weeks when administered.

11. The method according to claim 1, wherein the compound of formula (I) or a pharmaceutically acceptable salt thereof is administered to a patient at a dose corresponding to the mole equivalent of about 80 mg to about 160 mg of the free base of the compound of formula (I) per week during weeks when administered.

12. The method according to claim 1, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, naevi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, myeloproliferative disorders, lymphoproliferative disorders and myelodysplastic syndromes.

13. The method according to claim 1, wherein the neoplastic disease is one which is treatable by inhibition of PLK1 in addition to a treatment with a TTK inhibitor.

14. The method according to claim 1, wherein the neoplastic disease is a cancer, in particular a cancer selected from breast cancer, gastric cancer, colorectal cancer, liver cancer, endometrial cancer, ovarian cancer, esophageal cancer, lung cancer, Kaposi's sarcoma, cervical cancer, pancreatic cancer, melanoma, prostate cancer, bladder cancer and leukemia.

15. The method according to claim 1, wherein the neoplastic disease is triple negative breast cancer.
